SEQUENCE LISTING (4 sequences)

SEQ ID NO: 1 - DNA, 20 bp, synthetic construct
  source 1..20: mol_type = other DNA
  modified_base 1: mod_base = OTHER; note = PHOSPHORYLATION
gatcggaagagcacacgtct

SEQ ID NO: 2 - DNA, 33 bp, synthetic construct
  source 1..33: mol_type = other DNA
acactctttccctacacgacgctcttccgatct

SEQ ID NO: 3 - DNA, 58 bp, synthetic construct
  source 1..58: mol_type = other DNA
  modified_base 57..58: mod_base = OTHER; note = phosphorothioate bond
aatgatacggcgaccaccgagatctacactctttccctacacgacgctcttccgatct

SEQ ID NO: 4 - DNA, 66 bp, synthetic construct
  source 1..66: mol_type = other DNA
  modified_base 65..66: mod_base = OTHER; note = phosphorothioate bond
caagcagaagacggcatacgagatnnnnnnnngtgactggagttcagacgtgtgctcttccgatct

(19) United States
(12) Patent Application Publication
(10) Pub. No.: US 2026/0188525 A1
(43) Pub. Date: Jul. 2, 2026

(54) MICROBIOME MARKERS

(71) Applicant: AGENCY FOR SCIENCE, TECHNOLOGY AND RESEARCH, Singapore (SG)

(72) Inventors: Eileen PNG, Singapore (SG); Aarthi RAVIKRISHNAN, Singapore (SG); Kem Rei CHNG, Singapore (SG); Niranjan NAGARAJAN, Singapore (SG)

(73) Assignee: AGENCY FOR SCIENCE, TECHNOLOGY AND RESEARCH, Singapore (SG)

(21) Appl. No.: 19/101,169

(22) PCT Filed: Aug. 4, 2023
(86) PCT No.: PCT/SG2023/050542

§ 371 Date: Feb. 4, 2025

(30) Foreign Application Priority Data
Aug. 4, 2022 (SG) 10202250669E

(51) Int. Cl. G16H 50/70; A61K 45/06; C12Q 1/06; C12R 1/19; C12R 1/22; C12R 1/46; G16B 40/20; G16H 10/40; G16H 10/60; G16H 50/30

(52) U.S. Cl. CPC G16H 50/70; A61K 45/06; C12Q 1/06; G16B 40/20; G16H 10/40; G16H 10/60; G16H 50/30; C12R 2001/19; C12R 2001/22; C12R 2001/46

(57) ABSTRACT

This invention relates to microbiome markers for healthy aging. There is provided, inter alia, a method of predicting or determining healthy aging in an individual, the method comprising: (a) determining the presence or an amount of at least one microorganism in a sample representing the individual's gut microbiome, wherein the at least one microorganism is selected from Table 1A, wherein an enrichment of said microorganisms is indicative of healthy aging; and/or (b) determining the presence or an amount of at least one microorganism in a sample representing the individual's gut microbiome, wherein the at least one microorganism is selected from Table 1 B, wherein a depletion of said microorganisms is indicative of healthy aging.

### CROSS-REFERENCE TO RELATED APPLICATIONS

The present application is a National Stage Application of International Application No. PCT/SG2023/05042, filed Aug. 4, 2023, which claims priority to and the benefit of Singapore patent application Ser. No. 10202250669E, filed Aug. 4, 2022, both of which are herein incorporated by reference in their entireties.

The instant application contains a Sequence Listing which has been submitted electronically in XML format and is hereby incorporated by reference in its entirety. Said XML copy, created on May 30, 2025, is named 245866.000015_SL and is 6,296 bytes in size.

This invention relates to biomarkers. In particular, this invention relates to microbiome markers for healthy aging. This invention is a result of the Identification of key bacteria species in the gut microbiome of healthy aged subjects and their association with disease risk phenotypes.

Over the last few decades, economic changes and advances in healthcare systems have significantly improved life expectancies in Asia, and are expected to lead to a rapid shift in demographics by doubling the population of 60+ individuals by 2050 (from 19.7% in 2015 to 40%). This has been accompanied by a rising incidence of chronic diseases linked to aging, and corresponding socio-economic stress on healthcare systems across Asia. In the next 50 years, 20% of the world's population would be over 65 years. Although lifespan has increased, the elderly are spending an extended period of their remaining years in poor health and with disabilities. Hence, an ageing population would face a sizeable socioeconomic impact and pose a significant burden to the healthcare system. Interventions to promote healthy ageing have been encouraged to reduce disability adjusted life year (DALY). There is significant evidence that the gut microbiome can serve as biomarker to quantify biological age and is amenable for intervention to improve health and extend quality adjusted life year (QALY).

Aging is believed to be a complex, multi-factorial phenomenon with progressive decline in several physiological functions including in the gastrointestinal and immune system. Not surprisingly, many studies have therefore identified correlations between gut microbiome composition and age.

There is therefore an urgent need to identify lifestyle, dietary and pharmaceutical interventions that promote healthy aging in Asian populations.

The listing or discussion of an apparently prior-published document in this specification should not necessarily be taken as an acknowledgement that the document is part of the state of the art or is common general knowledge.

Any document referred to herein is hereby incorporated by reference in its entirety.

In an aspect of the invention, there is provided a method of predicting or determining healthy aging in an individual, the method comprising: (a) determining the presence or an amount of at least one microorganism in a sample representing the individual's gut microbiome, wherein the at least one microorganism is selected from Table 1A, wherein an enrichment of said microorganisms is indicative of healthy aging; and/or (b) determining the presence or an amount of at least one microorganism in a sample representing the individual's gut microbiome, wherein the at least one microorganism is selected from Table 1B, wherein a depletion of said microorganisms is indicative of healthy aging.

This invention provides for the following: showing the taxa and the pathways related to ageing; and associations with other phenotypic markers.

Tables 1A and 1B below provide the list of microorganisms that are associated with healthy aging. The enrichment and depletion are determined based on the β coefficient value obtained by the methods of the present invention where a positive value means an increase and a negative value means reduction in the abundance of the respective taxa.

TABLE 1A
microorganisms enriched with age

| | FDR-adjusted | β coefficient for age | |
|---|---|---|---|
| Enriched with age | p-value | [95% C.I.] | |
| Alistipes senegalensis | 7.77 × 10⁻⁵ | 19.61 | [10.92, 28.29] |
| Alistipes indistinctus | 1.00 × 10⁻² | 3.21 | [1.07, 5.32] |
| Alistipes unclassified | 5.97 × 10⁻⁵ | 2.48 | [1.41, 3.54] |
| Bacteroides xylanisolvens | 5.81 × 10⁻³ | 2.46 | [1.20, 3.73] |
| Bacteroidales bacterium | 5.97 × 10⁻⁵ | 1.61 | [0.91, 2.30] |
| ph8 | | | |
| Alistipes shahii | 0.10 × 10⁻² | 1.59 | [1.03, 2.15] |
| Bacteroides clarus | 0.50 × 10⁻² | 1.37 | [0.24, 2.50] |
| Parabacteroides merdae | 2.99 × 10⁻² | 0.95 | [−0.03, 1.93] |
| Parabacteroides distasonis | 4.01 × 10⁻² | 0.83 | [0.15, 1.50] |
| Bacteroides dorei | 6.46 × 10⁻⁵ | 0.78 | [0.51, 1.04] |

TABLE 1B
microorganisms depleted with age

| | FDR-adjusted | β coefficient for age | |
|---|---|---|---|
| Depleted with age | p-value | [95% C.I.] | |
| Bacteroides massiliensis | 2.52 × 10⁻² | −0.47 | [−0.86, −0.07] |
| Bifidobacterium | 6.72 × 10⁻⁶ | −1.76 | [−2.72, −0.8] |
| pseudocatenulatum | | | |
| Bilophila unclassified | 6.38 × 10⁻⁵ | −6.34 | [−9.55, −3.13] |
| Eubacterium hallii | 1.08 × 10⁻¹³ | −8.93 | [−11.75, −6.11] |
| Eubacterium rectale | 6.35 × 10⁻³ | −0.75 | [−1.06, −0.44] |
| Eubacterium ventriosum | 5.97 × 10⁻⁵ | −5.99 | [−8.60, −3.4] |
| Lachnospiraceae bacterium | 7.34 × 10⁻⁵ | −4.48 | [−6.45, −2.51] |
| 1 1 57FAA | | | |
| Parasutterella | 3.68 × 10⁻⁴ | −19.26 | [−28.68, −9.84] |
| excrementihominis | | | |
| Ruminococcus obeum | 5.12 × 10⁻¹⁴ | −11.08 | [−13.66, −8.49] |
| Ruminococcus sp 5 1 | 5.72 × 10⁻¹⁸ | −1.46 | [−2.34, −0.58] |
| 39BFAA | | | |
| Ruminococcus torques | 6.33 × 10⁻³ | −1.27 | [−2.06, −0.48] |

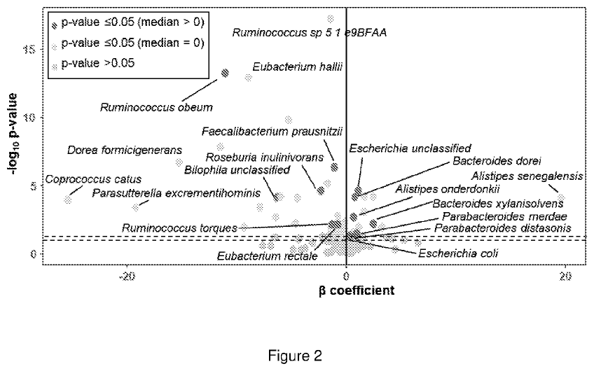

Tables 1A and 1B—Aging associated core gut microbial species in Asians. Results are based on FDR-adjusted p-value≤0.05 using GLM test with co-variate adjustment. The β coefficient estimates strength of association as change in age (in years) for every 1% increment of taxa abundance. These are the values of β estimate, Standard Error, FDR-adjusted p-value associated with age. The y-axis on the FIG. 2 is −log₁₀ of FDR-adjusted p-values.

By “healthy aging”, it is meant to refer to the definition of healthy ageing according to the World Health Organisation (WHO) being “Healthy Ageing is the process of developing and maintaining the functional ability that enables wellbeing in older age.” https://www.who.int/ageing/en/.

The dataset present in Tables 1A and 1B (and also in Tables 7A and 7B below), and hence the determination of the microorganisms that are shown to be enriched or depleted with age, are based on individuals that are ageing healthily, i.e. individuals that report no known ailments or disease or conditions. Gut microbiome profiles of these individuals are obtained and the values in the tables are then computed. The values shown in the tables are obtained using GLM test with co-variate adjustment. The β coefficient estimates strength of association as change in age (in years) for every 1% increment of taxa abundance. These are the values of β estimate, Standard Error, FDR-adjusted p-value associated with age. The y-axis on the FIG. 2 is −log₁₀ of FDR-adjusted p-values. The microorganisms that are shown to be enriched with age show positive β estimate values while those microorganisms that are shown to be depleted with age show negative β estimate values. The relative abundances of these microorganisms can be measured through sequencing technologies.

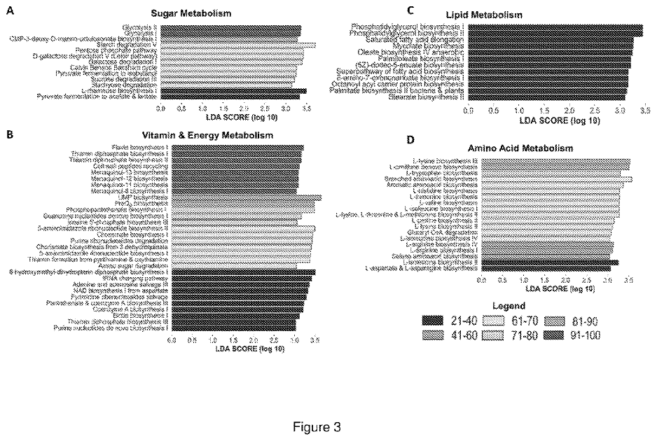

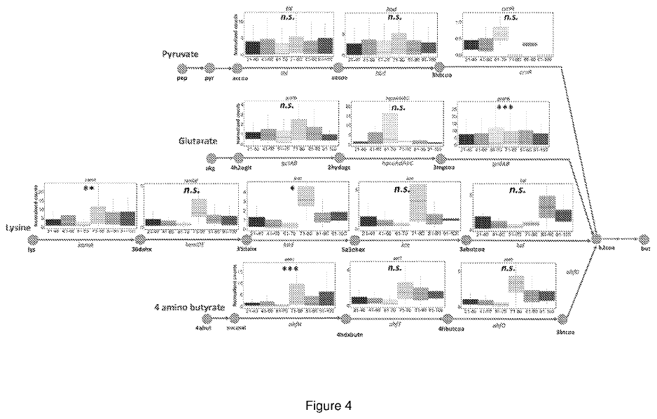

There are four different pathways for the production of butyrate as shown in FIG. 4. The conventional pathway for butyrate production involving pyruvate as a substrate is used by many of the core gut microorganisms such as Bacteroides dorei, Bacteroides xylanisolvens, Bacteroides massiliensis, which are depleted in healthy ageing individuals (Tables 1B, 7B). The inventors of this invention have found that this reduction is compensated by an enrichment in Alistipes species including Alistipes senegalensis, Alistipes indistinctus and Alistipes unclassified (Tables 1A, 7A), which produce butyrate through an alternate pathway using Lysine as the substrate (FIG. 4). Lysine biosynthesis is also enriched in the elderly as shown in FIG. 3, indicating that there is a switch in butyrate producing pathways.

In an embodiment of the invention, there is provided a method of predicting or determining healthy aging in an individual wherein the method comprises determining the presence or an amount of Alistipes species wherein an enrichment of Alistipes species is indicative of healthy aging.

Similarly, in another embodiment of the invention, there is provided a method of promoting healthy ageing by increasing the levels of Alistipes species in an individual. By “promoting” healthy aging, it is meant to allow individuals to achieve a healthy body while aging.

By “microorganisms”, it is meant include any wild type or mutant strains or genetically transformed strains. These mutants or genetically transformed strains can be strains wherein one or more endogenous gene(s) of the parent strain has (have) been mutated, for instance to modify some of their metabolic properties (e.g., their ability to ferment sugars, their resistance to acidity, their survival to transport in the gastrointestinal tract, their post-acidification properties or their metabolite production). They can also be strains resulting from the genetic transformation of the parent strain to add one or more gene(s) of interest, for instance in order to give to said genetically transformed strains additional physiological features, or to allow them to express proteins of therapeutic or vaccinal interest that one wishes to administer through said strains. These mutants or genetically transformed strains can be obtained from the parent strain by means of conventional techniques for random or site-directed mutagenesis and genetic transformation of bacteria, or by means of the technique known as “genome shuffling”.

By “enriching” or “enrichment” and “depleting” or “depletion”, it is meant to refer to any increase or reduction in the amounts of the relevant microorganism in an average individual respectively.

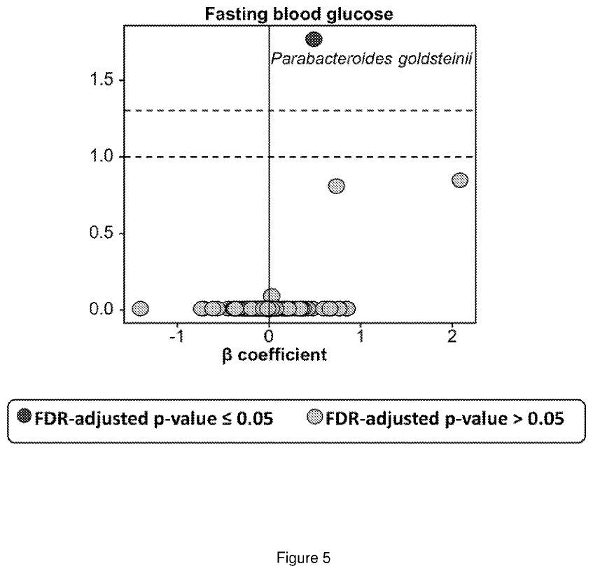

In another aspect of the invention, there is provided a method of predicting or determining an increased fasting blood sugar level in an individual, the method comprising determining the presence or an amount of microorganisms in a sample representing the individual's gut microbiome, wherein the microorganism is Parabacteroides goldsteinii, wherein an enrichment of said microorganisms is indicative of an increased fasting blood sugar level. FIG. 5 shows the data to support this aspect of the invention.

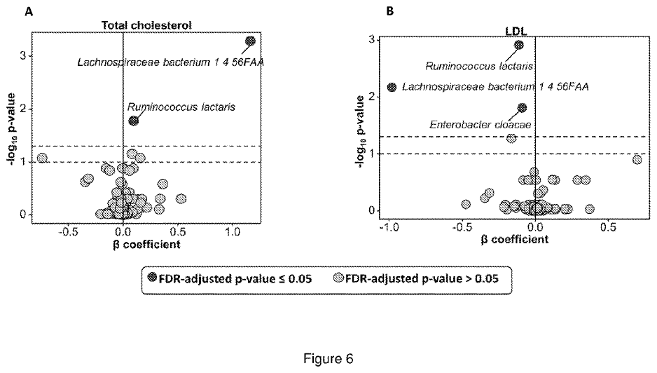

In another aspect of the invention, there is provided a method of predicting or determining total cholesterol levels in an individual, the method comprising determining the presence or an amount of microorganisms in a sample representing the individual's gut microbiome, wherein the microorganisms are Lachnospiraceae bacterium 14 56FAA and Ruminococcus lactaris, wherein an enrichment of said microorganisms is indicative of a higher than normal level of total cholesterol level. By “total cholesterol”, it includes LDL and HDL. FIG. 6 shows the data to support this aspect of the invention. FIG. 6A shows the data with regards to total cholesterol, while FIG. 6B shows the data with regards to LDL. Analysis with LDL levels revealed the opposite trend, with the Lachnospiraceae species (β=−0.98, FDR-adjusted p-value=1.21*10\hat{ }−3) and R. lactis (B=−0.11, FDR-adjusted p-value=6.74*10\hat{ }−3) being negatively associated. Further, we also found Enterobacter cloacae (B=−0.09, FDR-adjusted p-value=1.55*10\hat{ }−2) to be negatively associated with LDL levels.

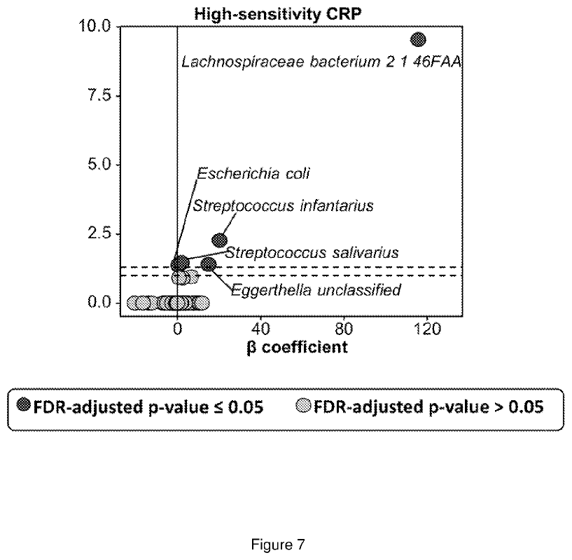

In another aspect of the invention, there is provided a method of predicting or determining inflammation associated with high-sensitivity C-reactive protein (HSCRP) in an individual, the method comprising determining the presence or an amount of microorganisms in a sample representing the individual's gut microbiome, wherein the microorganisms are Lachnospiraceae bacterium 2 1 46FAA, Streptococcus infantarius, Streptococcus salivarius, Eggerthella unclassified and Escherichia coli, wherein an enrichment of said microorganisms is indicative of inflammation. FIG. 7 shows the data to support this aspect of the invention.

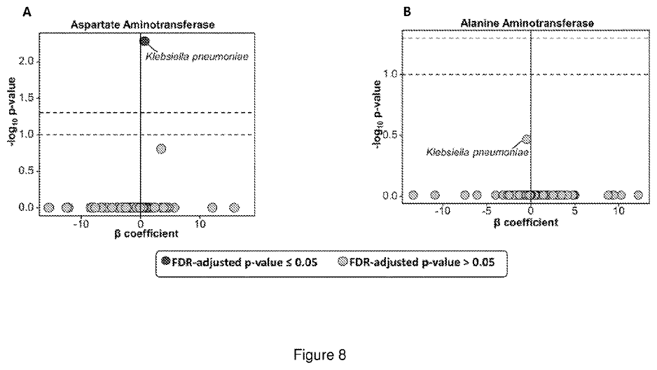

In another aspect of the invention, there is provided a method of predicting or determining hepatic health in an individual, the method comprising determining the presence or an amount of Klebsiella pneumoniae in a sample representing the individual's gut microbiome, wherein an enrichment of said microorganism is indicative of hepatic disease. FIG. 8 shows the data to support this aspect of the invention.

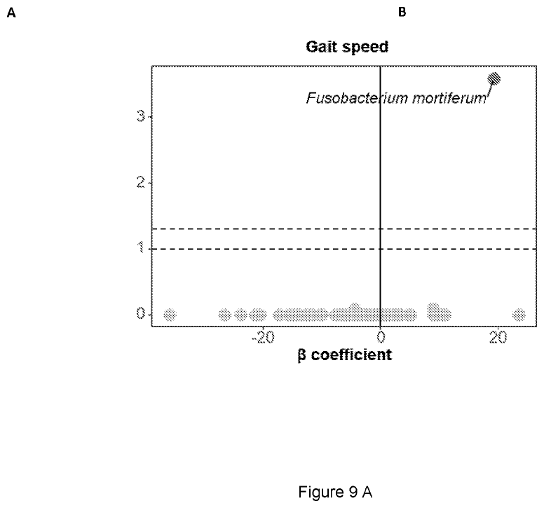

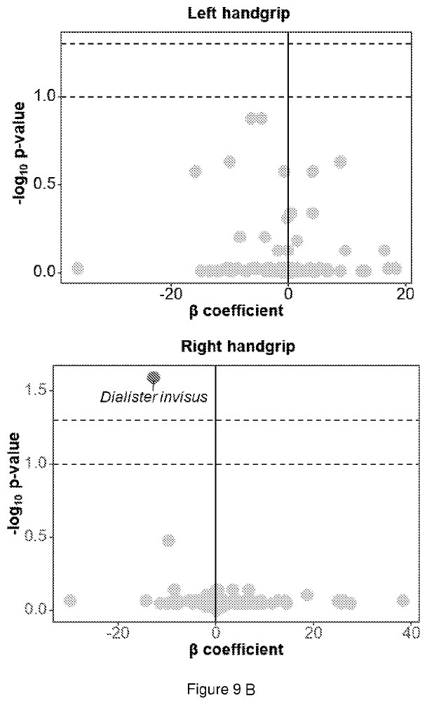

In another aspect of the invention, there is provided a method of predicting or determining physical strength or weakness in a subject, the method comprising determining the presence or an amount of Fusobacterium mortiferum in a sample representing the individual's gut microbiome, wherein an enrichment of said microorganism is indicative of physical strength. FIG. 9A shows the data to support this aspect of the invention.

In another aspect of the invention, there is provided a method of predicting or determining physical strength or weakness in a subject, the method comprising determining the presence or an amount of Dialister invisus in a sample representing the individual's gut microbiome, wherein a depletion of said microorganism is indicative of weakness. FIG. 9B shows the data to support this aspect of the invention.

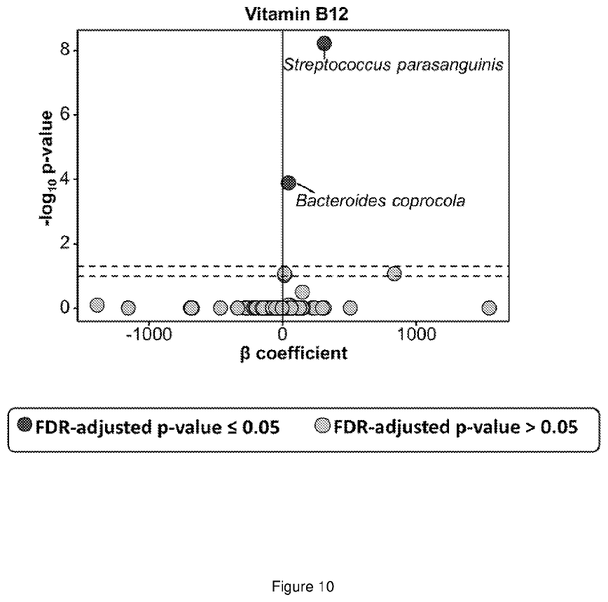

In another aspect of the invention, there is provided a method of predicting or healthy levels of vitamin B12 in a subject, the method comprising determining the presence or an amount of Streptococcus parasanguinis or Bacteroides coprocola in a sample representing the individual's gut microbiome, wherein an enrichment of said microorganism is indicative of healthy levels of vitamin B12. FIG. 10 shows the data to support this aspect of the invention. Healthy levels of vitamin B12 may include a value range of between 160 to 950 picograms per milliliter (pg/mL), or 118 to 701 picomoles per liter (pmol/L). Values of less than 160 μg/mL (118 μmol/L) are a possible sign of a vitamin B12 deficiency. People with this deficiency are likely to have or develop symptoms.

In another aspect of the invention, there is provided a method of promoting healthy ageing in a subject, the method comprising administering to a patient a composition that improves intestinal flora by: (a) enriching at least one microorganism selected from Table 1A, and (b) reducing or suppressing at least one microorganism selected from Table 1B.

In another aspect of the invention, there is provided a pharmaceutical composition comprising an isolated microorganism for use in promoting healthy ageing in a subject, wherein the isolated microorganism is selected from Table 1A.

In another aspect of the invention, there is provided a composition or pharmaceutical composition for promoting healthy ageing, the composition comprising an agent for: (a). enriching at least one microorganism selected from Table 1A; and/or (b). reducing or suppressing at least one microorganism selected from Table 1B.

In another aspect of the invention, there is provided a use of a composition comprising at least one of or a combination of microorganisms selected from Table 1A in the manufacture of a medicament for promoting healthy ageing.

By “composition”, it is meant to include any “synthetic composition” or formulation that is artificially made and not naturally occurring. Any such suitable formulation would include any process of isolating, purifying and manufacture to ensure said formulation is safe for human consumption. For example, the synthetic composition may be a probiotic or a pharmaceutical formulation, or a food product.

The term “probiotic”, “food product” and “pharmaceutical composition” have generally accepted definitions. For example, probiotics may be defined as live microorganisms thought to be healthy for the host organism; digestive enzymes may be defined as enzymes that break down polymeric macromolecules into their smaller building blocks in order to facilitate their absorption by the body; dietary supplements may be defined as a preparation intended to supplement the diet and provide nutrients that may be missing or may not be consumed in sufficient quantities in a human's diet.

The selected microorganisms of the invention may be in a liquid culture or dried form for administration. The drying of bacterial strains after production by fermentation is known to the skilled person. See for example, EP 0 818 529 (SOCIETE DES PRODUITS NESTLE), which is incorporated by reference in its entirety, where a drying process of pulverization is described. In some embodiments, the microorganisms may be lyophilized, pulverized and powdered. Usually, bacterial microorganisms are concentrated from a medium and dried by spray drying, fluidised bed drying, lyophilisation (freeze drying) or other drying process. Micro-organisms can be mixed, for example, with a carrier material such as a carbohydrate such as sucrose, lactose or maltodextrin, a lipid or a protein, for example milk powder during or before the drying.

The bacterial strain need not necessarily be present in a dried form. It may also be suitable to mix the bacteria directly after fermentation with a food product and, optionally, perform a drying process thereafter. Such an approach is disclosed in PCT/EP02/01504, which is incorporated by reference in its entirety. Likewise, a probiotic composition of the invention may also be consumed directly after fermentation. Further processing, for example, for the sake of the manufacture of convenient food products, is not a precondition for the beneficial properties of the bacterial strains provided in the probiotic composition.

The compositions according to the present invention may be enterally consumed in any form. They may be added to a nutritional composition, such as a food product. On the other hand, they may also be consumed directly, for example in a dried form or directly after production of the biomass by fermentation.

According to the subject invention, the bacterial strain(s) can be provided in an encapsulated form in order to ensure a high survival rate of the micro-organisms during passage through the gastrointestinal tract or during storage or shelf life of the product.

The compositions of the subject invention may, for example, be provided as a probiotic composition that is consumed in the form of a fermented, dairy product, such as a chilled dairy product, a yogurt, or a fresh cheese. In these later cases, the bacterial strain(s) may be used directly also to produce the fermented product itself and has therefore at least a double function: the probiotic functions within the context of the present invention and the function of fermenting a substrate such as milk to produce a yogurt.

If the bacterial strain is added to a nutritional formula, the skilled person is aware of the possibilities to achieve this. Dried, for example, spray dried bacteria, such as obtainable by the process disclosed in EP 0 818 529 (which is incorporated herein by reference in its entirety) may be added directly to a nutritional formula in powdered form or to any other food product. For example, a powdered preparation of the bacterial strain(s) of the invention may be added to a nutritional formula, breakfast cereals, salads, a slice of bread prior to consumption.

In various embodiments, the microorganism composition is a liquid culture that may be administered to a subject.

Bacterial strain(s) of the invention may be added to a liquid product, for example, a beverage or a drink. If it is intended to consume the bacteria in an actively-growing state, the liquid product comprising the bacterial strain(s) should be consumed relatively quickly upon addition of the bacteria. However, if the bacteria are added to a shelf-stable product, quick consumption may not be necessary, so long as the bacterial strain(s) are stable in the beverage or the drink.

WO 98/10666, which is incorporated herein by reference in its entirety, discloses a process of drying a food composition and a culture of probiotic bacteria conjointly. Accordingly, the subject bacterial strain(s) may be dried at the same time with juices, milk-based products or vegetable milks, for example, yielding a dried product already comprising probiotics. This product may later be reconstituted with an aqueous liquid.

By “food product”, it is also meant to include any food supplements made from compounds usually used in foodstuffs, but which is in the form of tablets, powder, capsules, potion or any other form usually not associated with aliments, and which has beneficial effects for one's health. It is meant to also include any “functional food” which has beneficial effects for one's health in addition to providing nutrients. In particular, food supplements and functional food can have a physiological effect—for the prophylaxis, amelioration or treatment of a disease, for example a chronic disease.

The composition can be a pharmaceutical composition or a nutritional composition. In various embodiments, the composition is a nutritional composition such as a food product (including a functional food) or a food supplement.

Nutritional compositions which can be used according to the invention include dairy compositions, preferably fermented dairy compositions. The fermented compositions can be in the form of a liquid or in the form of a dry powder obtained by drying the fermented liquid. Examples of dairy compositions include fermented milk and/or fermented whey in set, stirred or drinkable form, cheese and yoghurt. The fermented product can also be a fermented vegetable, such as fermented soy, cereals and/or fruits in set, stirred or drinkable forms. Nutritional compositions which can be used according to the invention also include baby foods, infant milk formulas and infant follow-on formulas. In various embodiments, the fermented product is a fresh product. A fresh product, which has not undergone severe heat treatment steps, has the advantage that the bacterial strains present are in the living form.

In various embodiments, the pharmaceutical composition is formulated for oral administration. The pharmaceutical composition may comprise a coating, optionally wherein the coating is an enteric coating. The coating material comprises at least one of a saccharide, a polysaccharide, and a glycoprotein extracted from at least one of a plant, a fungus, and a microbe, optionally wherein the at least one of a saccharide, a polysaccharide, and a glycoprotein includes one or more of corn starch, wheat starch, potato starch, tapioca starch, cellulose, hemicellulose, dextrans, maltodextrin, cyclodextrins, inulins, pectin, mannans, gum arabic, locust bean gum, mesquite gum, guar gum, gum karaya, gum ghatti, tragacanth gum, funori, carrageenans, agar, alginates, chitosans, or gellan gum.

In various embodiments, the pharmaceutical composition is formulated with a germinant.

The probiotic ingredients of the composition may be present in an effective dose. For example, at the time of manufacture, the probiotic ingredients may total at least 6×10⁹ colony forming units (cfu) and may include at least 13×10⁹ cfu of probiotics or more. In various embodiments, the probiotic ingredients total at least 13×10⁹ cfu of probiotics. In various embodiments, the probiotic ingredients total at least 14×10⁹ cfu of probiotics. A colony forming unit (cfu) is generally accepted as a measure of viable bacterial or fungal numbers. Such quantity of probiotic ingredient may facilitate providing a consumer with an effective dose of probiotics at the time of ingestion, as the inventor has realized that probiotics may be destroyed during storage due to undesirable environments (e.g., temperature extremes) and other reasons. In various embodiments, the composition is formulated in a dosage form at least about 1×10⁴ colony forming units of bacteria.

In various embodiments, the consumption or administration of a dose of between about 10⁸ and about 10¹¹ colony forming unit (CFU) of at least one of or any combination of microorganisms set out in Table 1. In other embodiments, it could be between about 10⁸ and about 10⁹. Alternatively, it could be between about 10⁹ and about 10¹⁰ colony forming unit (CFU) and in an alternative embodiment between about 10¹⁰ and about 10¹¹ colony forming unit (CFU). In various embodiments least 1, 2, 3, or 4 doses are provided within a 24 hour time period. It is further preferred that the daily dosage regimen is maintained for at least about 1, 2, 3, 4, 5, 6 or 7 days, or in alternative embodiment for at least about 1, 2, 3, 4, 5, 6 or 7 weeks.

The composition of the invention may be incorporated into a food product, e.g. yoghurt. Alternatively, to facilitate protection of the composition, capsules comprising the composition may be and are preferably stored in blister packs. That is, the blister packs may seal the capsule from a surrounding environment and thus, extend the life of the effective ingredients of the composition.

Oral delivery of the composition is accomplished via a 2 to 4 ounces emulsion or paste mixed with an easy to eat food such as a milk shake or yoghurt. The microencapsulated bacterial probiotic and prebiotic can be administered along with the mixture of sorbents in the emulsion or paste or separately in a swallowable gelatin capsule.

A mathematical model of solute transport of oral sorbents has been developed based on the diffusion controlled solute flux into the intestinal lumen followed by physical binding or chemical trapping (Gotch et al. Journal of Dialysis 1976-1977 1 (2): 105-144). This model provides the theoretical basis of solute removal through the gut.

Any method of using the composition may be used as desired by consumers of the composition. A particularly advantageous program may be to take a single capsule of the composition on a daily basis until the effects of the gut microbiome dysbiosis is reduced or eliminated.

In another aspect of the invention, there is provided a method of predicting the likelihood of healthy aging in an individual, the method comprising: (a) determining a gut microbiome signature of the individual by determining an amount of, or presence or absence of, each microorganism in a group of microorganisms present in a sample obtained from the individual; and (b) applying a prediction model to assess the gut microbiome signature with respect to a gut profile representative of an individual that is aging healthily, wherein the prediction model is trained using a dataset of microbiome profiles of a plurality of individuals who are aging healthily and said profile comprises: (i) an enrichment of at least one microorganism selected from Table 1A; and/or (ii) a depletion of at least one microorganism selected from Table 1B.

In an embodiment, the prediction model comprises a machine learning probability model.

In another aspect of the invention, there is provided a computer readable storage medium comprising computer readable instructions operable when executed by a computer to predict the likelihood of healthy aging in an individual, the computer readable instructions configured to perform a method according to any one of the above aspects of the invention.

In another aspect of the invention, there is provided an apparatus or system comprising: (a) a receiving unit configured to receive a dataset of values representing a gut microbiome signature of an individual by determining an amount of, or presence or absence of, each microorganism in a group of microorganisms present in a sample obtained from the individual; and (b) a processor configured to process a prediction model to assess the gut microbiome signature with respect to a gut profile representative of good gut health to obtain a likelihood of healthy aging in the individual, wherein the prediction model is trained using a dataset of microbiome profiles of a plurality of individuals who are aging healthily and said profile comprises: (i) an enrichment of at least one microorganism selected from Table 1A; and/or (ii) a depletion of at least one microorganism selected from Table 1B.

As will be understood by those skilled in the art, such a prediction is usually not intended to be correct for 100% of the subjects to be assessed by the present invention. The method for predicting a subject's likelihood of recovery, however, requires that the prediction to be at the likelihood of recovery, or not, is correct for a statistically significant portion of the subjects (e.g. a cohort in a cohort study). Whether a portion is statistically significant can be determined without further ado by the person skilled in the art using various well known statistic evaluation tools, e.g., determination of confidence intervals, p-value determination, Student's t-test, Mann-Whitney test etc. Details may be found in Dowdy and Wearden, Statistics for Research, John Wiley & Sons, New York 1983. Exemplary confidence intervals are at least 90%, at least 95%, at least 97%, at least 98% or at least 99%. The p-values may include 0.1, 0.05, 0.01, 0.005, or 0.0001.

The identification of the microorganisms, and their associations, of the present invention have led to the use of the automatic or machine learning to create a machine learning model for predicting the likelihood of healthy aging (and other phenotypic traits and metabolic pathways) in a subject.

In various embodiments, the prediction model comprises a machine learning probability model. The prediction model may comprise a random forest classification model, or a linear discriminant analysis model, or a sparse logistic regression model, or a conditional inference tree model. In various embodiments, one measure of dysbiosis may be arriving at a diversity score and based on Shannon entropy, i.e. for relative abundances pᵢ for species I, sum over all i of pᵢ log(pᵢ) may be computed. The prediction model may be any model including a decision tree.

In various embodiments, the gut microbiome signature of the subject is determined using a statistical analysis. These are based on statistical model (GLM) derived using relative abundances of organisms and hence it is very difficult to give a number to quantify. β coefficients obtained in the present invention is a direct output from the GLM model applied in the method described in this invention. B values are derived from the Generalized Linear Models (GLM) that were built using the microbiome abundances and the values of clinical parameters (one at a time). For example, to find out the association between fasting blood glucose and the microbiome, a GLM using these values is built. The model provides the β values, p-values for each of the organisms and the standard error. A multiple hypothesis correction using p-values to get the FDR-adjusted p-value is then carried out. A negative β coefficient may refer to a particular microorganism being “reduced abundance” or “depleted”, while a positive β coefficient may refer to a particular microorganism being “abundance” or “enriched”.

β coefficients can be interpreted as “for every unit change (%) in the relative abundance of a microorganism, response (clinical phenotypes such as fasting blood glucose, hsCRP, Total cholesterol levels) will have a change by an amount B, provided the other confounding factors are controlled”. These relative abundance values are derived from Illumina sequencing data which are given as input to the model. B values are the output from GLM. Confounding factors are those factors which have the potential to influence our results. Overall, these factors include Age, Fasting blood Glucose, TCHOL, HDL, LDL, BMI, gender. These are the values of β estimate, Standard Error, FDR-adjusted p-value of various clinical phenotypes that have been shown in FIGS. 5-10

In another aspect of the invention, there is provided an assay kit for use in the method according to any one of above aspects of the invention, and additionally comprising instructions to be used in the method.

Advantageously, the invention identifies key bacteria species associated with healthy ageing, the genes involved in maintaining butyrate production, the key bacteria species as potential intervention targets/markers for frailty and metabolic disease risk phenotypes, and an independent set of microbiome indicators for inflammation, infection, and early markers of inflammation. The invention shows that butyrate production from pyruvate may be compensated by the conversion of lysine to butyrate.

This invention is based on shotgun metagenomics. Further, the associations between the microorganisms and the various phenotypic traits or metabolic pathways are made at a species level. The results obtained here are at a higher level of resolution compared to other studies that had been carried out previously.

In order that the present invention may be fully understood and readily put into practical effect, there shall now be described by way of non-limitative examples only preferred embodiments of the present invention, the description being with reference to the accompanying illustrative figures.

In the Figures:

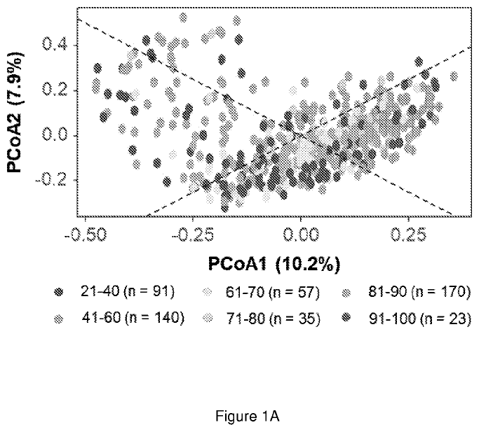

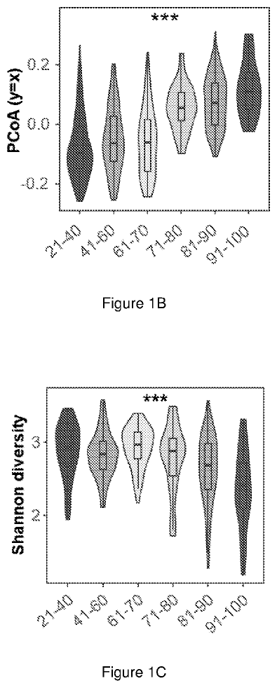

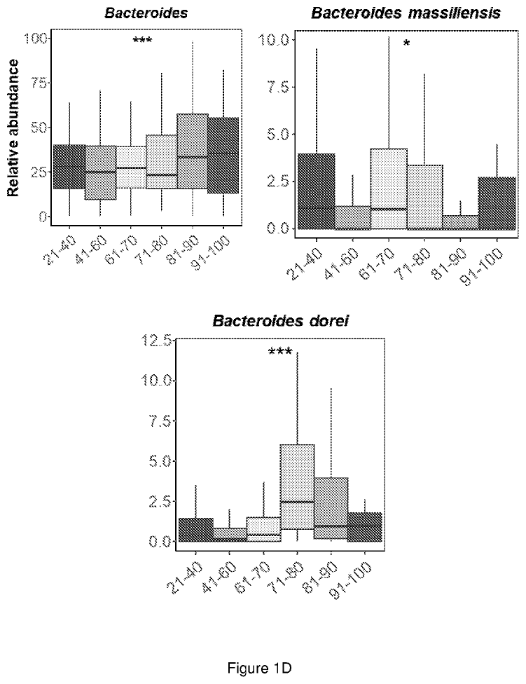

FIG. 1 shows aging-associated shifts in gut microbiome richness and relative abundance of key species. (A) Principal coordinates analysis (PCoA) plot based on species-level Bray-Curtis dissimilarity of gut microbiome profiles across age groups after batch correction. Dashed lines indicate the y=x and y=−x axes. (B-C) Violin plots showing the distribution across age groups of (B) PCoA1 and PCoA2 values projected on the y=x axes and (C) Shannon diversity indices. The symbols ‘**’ and ‘***’ represent p-value<0.05 and p-value<0.005 (Ordinal logistic test), respectively. (D) Relative abundance boxplots for the Bacteroides genus, and corresponding species that are identified to be associated significantly with age. The symbols “n.s.”, “*” and “***” represent p-value>0.05, p-value≤0.05 and p-value<0.005, respectively (FDR-adjusted; GLM test for association with age).

FIG. 2 is a volcano plot showing the β coefficient on the x-axis and FDR-adjusted −log₁₀ p-values (GLM test for taxa association with age) on the y-axis. Points for statistically significant taxa are colored in the volcano plot as red for median abundance greater than zero and green otherwise, with non-significant taxa shown with grey dots.

FIG. 3 shows associations of microbial metabolic pathways across age groups. Boxplot showing pathways that are significantly associated and enriched specifically in different age groups (p-value≤0.05) based on LEfSe analysis. Pathways were grouped into the broad categories of (A) Sugar metabolism, (B) Vitamin and energy metabolism, (C) Lipid metabolism and (D) Amino acid metabolism. The effect size is depicted in the form of an LDA Score.

FIG. 4 shows a differential representation of butyrate synthesis pathways in gut metagenomes across age groups. Pathway diagram where nodes represent major metabolites involved in various steps of butyrate production (but) starting from 4 different precursors (pyruvate, glutarate, lysine and 4-aminobutyrate), and edges are labelled with the genes involved in the conversion. Boxplots present normalized counts for the corresponding gene across different age groups. The labels “n.s.”, “*”, “**” and “***” represent p-value>0.05, p-value≤0.05, p-value<0.01 and p-value<0.001, respectively (FDR-adjusted GLM test for gene association with age).

FIGS. 5 to 10 show microbiome associations with key clinical markers. Volcano plots showing the β coefficient on the x-axis and the FDR-adjusted −log₁₀ p-values on the y-axis based on GLM test for gut microbial associations with clinical markers. Points in all plots are coloured with black for p-value≤0.05 and grey otherwise. Dotted lines represent the thresholds of p-value=0.05 and p-value=0.1.

FIG. 5 is a volcano plot showing the β coefficient on the x-axis and the y-axis is the FDR-adjusted −log₁₀ P values for taxa association with fasting blood glucose after age and other co-variates adjustment.

FIG. 6 are volcano plots showing the β coefficient on the x-axis and the y-axis is the FDR-adjusted −log₁₀ P values from the GLM test for taxa association with (A) Total cholesterol and (B) LDL levels after age and other co-variates adjustment.

FIG. 7 is a volcano plot showing the β coefficient on the x-axis and the y-axis is the FDR-adjusted −log₁₀ P values for taxa association with hs-CRP after age and other co-variates adjustment.

FIG. 8 are volcano plots showing the β coefficient on the x-axis and the y-axis is the FDR-adjusted −log₁₀ P values from the GLM test for taxa association (A) AST and (B) ALT levels after age and other co-variates adjustment.

FIG. 9 are volcano plots showing the β coefficient on the x-axis and the y-axis is the FDR-adjusted −log₁₀ P values from the GLM test for taxa association (A) gait speed and (B) right handgrip strength after age and other co-variates adjustment.

FIG. 10 are volcano plots showing the β coefficient on the x-axis and the y-axis is the FDR-adjusted −log₁₀ P values from the GLM test for taxa association Vitamin B12 after age and other co-variates adjustment.

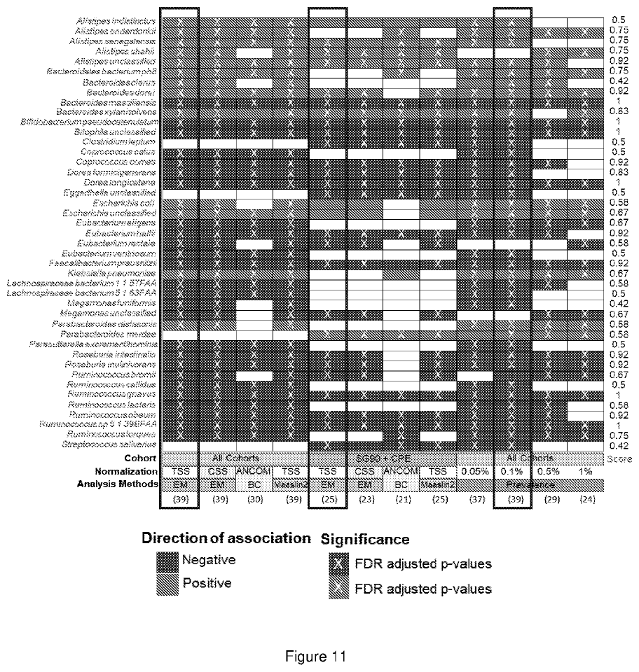

FIG. 11 shows data obtained from assessing the reproducibility and robustness of microbial associations with age. Heatmap shows microbial species associations with age, where (i) reproducibility was assessed through 3 distinct primary analysis approaches (black boxes) and (ii) robustness was assessed by varying their parameters, normalization and analysis settings (all columns; see method described below). The robustness score (last column) captures the frequency with which a taxa was observed to be significantly associated with age across all tested methods and settings (FDR adjusted p-value<0.05). Note that all but three associations had robustness score greater than or equal to 50%. Numbers in parentheses below each column indicate the number of significant associations for each method. Red and blue colored cells represent positive and negative associations, respectively. Colored cells marked with ‘X’ correspond to FDR adjusted p-value<0.05, while cells without an ‘X’ correspond to unadjusted p-value<0.05.

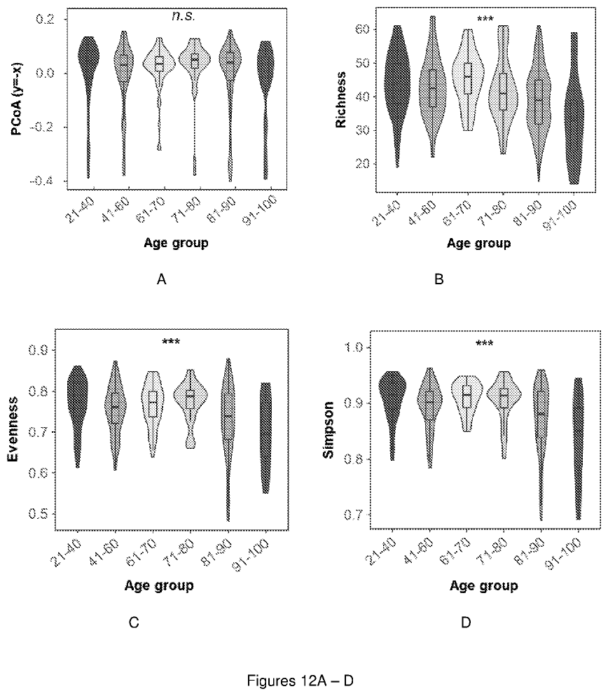

FIG. 12 shows variation in gut microbiome beta and alpha diversity metrics across age groups. (A) Violin plot showing the variation of beta diversity on the y=−x axes corresponding to the PCoA plot in FIG. 1A. Violin plots showing species-level (B) Richness, (C) Evenness and (D) Simpson diversity index, across age groups. All three indices were found to be significantly different between age groups (ordinal logistic test; “***”: p-value<0.001). Data points that are either less than Q1−1.5×IQR or more than Q3+1.5×IQR, where Q1, Q3 and IQR refers to the first quartile, third quartile and interquartile range, respectively, were removed for this analysis to reduce the impact of outliers.

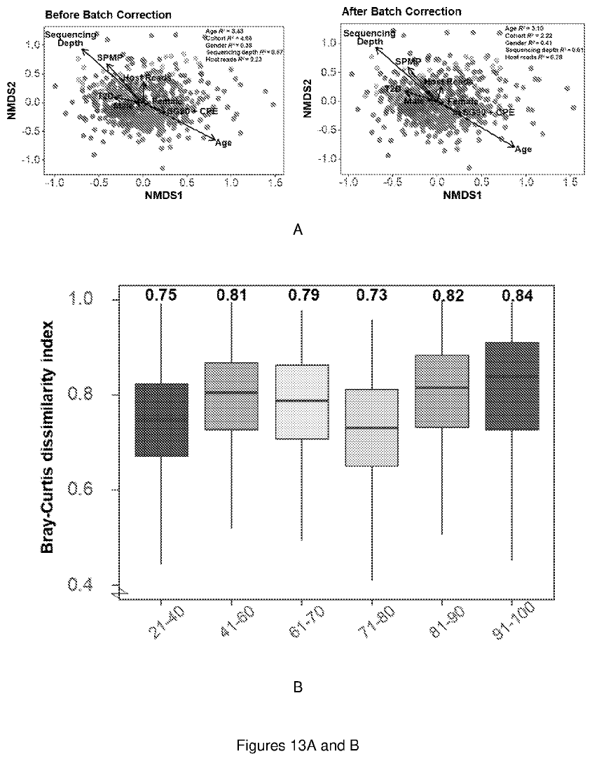

FIG. 13 shows variation of beta diversity metrics. (A) Nonmetric Multidimensional Scaling (NMDS) plots showing the distribution of samples for each cohort before (top) and after (bottom) batch correction. Vectors for each of the confounding variables were obtained through envfit analysis demonstrating the relationship between the ordination axes and the variables. Length of the vector indicates strength of the relationship, and the direction points to the steepest increase corresponding to the variable. Inset values for R² are from PERMANOVA analysis using Bray-Curtis dissimilarity. (B) Box plots showing the species-level Bray-Curtis dissimilarity index across age groups. In all boxplots, the center line represents the median, box limits represent upper and lower quartiles, and whiskers represent minimum and maximum values. Median values are shown on top of the plot.

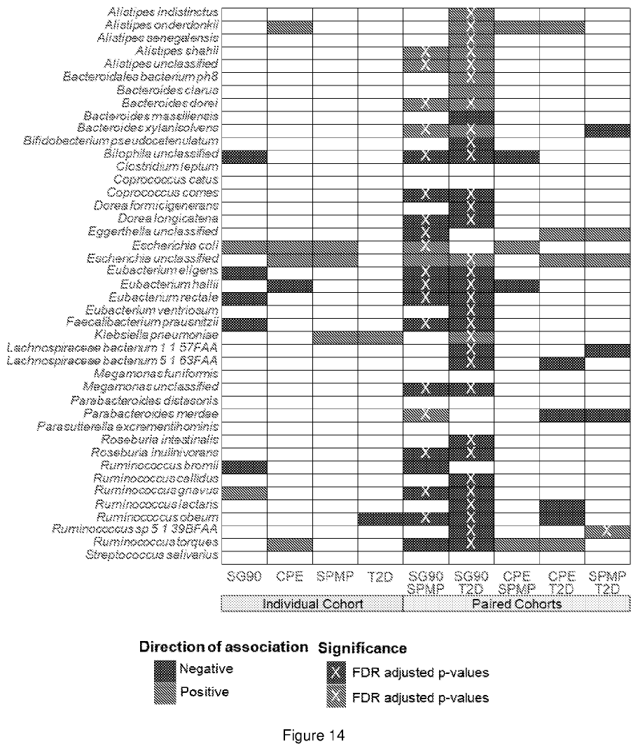

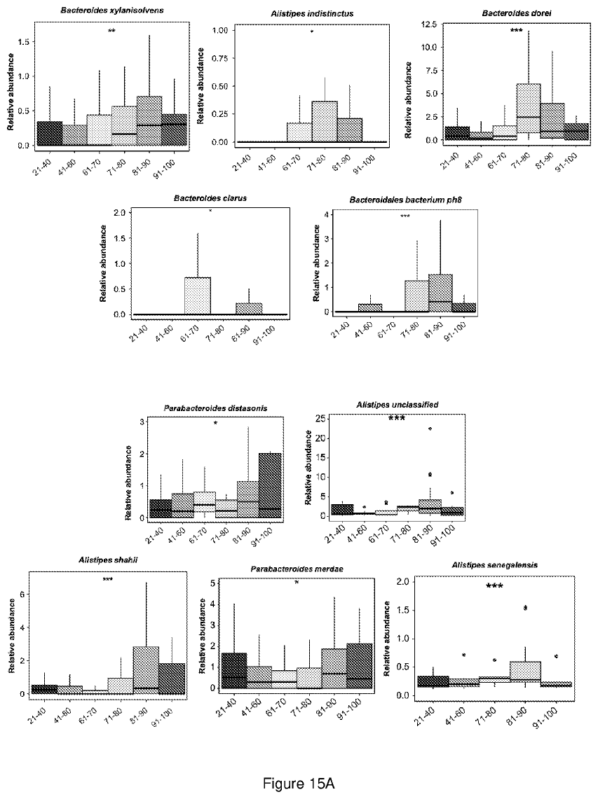

FIG. 14 shows the associations within individual and paired cohorts. Heatmap showing microbial species associations with age identified using independent and paired cohort analysis. Red and blue colored cells represent positive and negative associations, respectively. Colored cells marked with ‘X’ correspond to FDR adjusted p-value<0.05, while cells without an ‘X’ correspond to unadjusted p-value<0.05. As can be seen here, while some associations are identified in individual cohorts (n=13), joint analysis of SG90 with a cohort of younger individuals is essential to identify many more associations (n=37). These association are unlikely to be purely a function of batch effects as very few associations are detected when other pairs of cohorts are analyzed together (e.g. n=4, using CPE and SPMP), where these associations are also not significant after FDR adjustment.

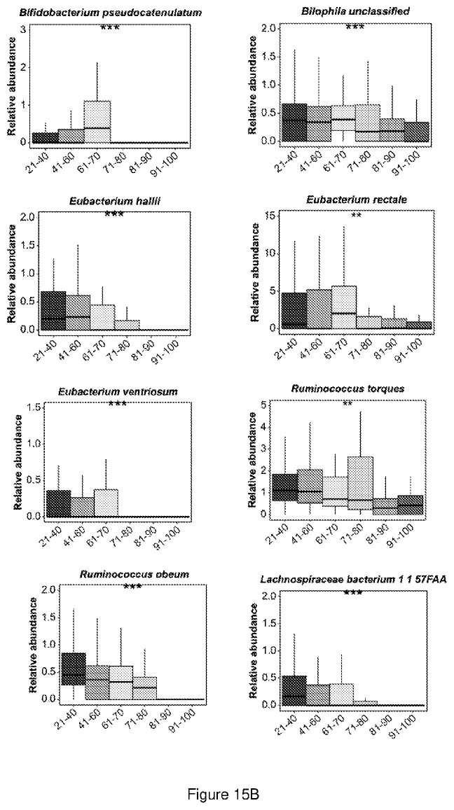

FIG. 15 shows the relative abundance across age groups for key gut microbial species associated with aging. Boxplots showing relative abundances across age groups for (A) species whose abundances increase with age and (B) species whose abundances decrease with age. “*” (p-Median FDR-adjusted p-values from the 3 primary analysis approaches are denoted by “*” value<0.05) and “***” (p-value<0.001). In all boxplots, the center line represents the median, box limits represent upper and lower quartiles, and whiskers represent minimum and maximum values (outlier points are not included in the visualization).

FIG. 16 shows the microbial co-occurrence patterns in elderly and young Asian subjects. Nodes in the network represent microbial species and edges represent significant Spearman correlations between relative abundances of species corresponding to adjacent nodes (|r|≥0.1, p-value<0.05). The network on the left (Elderly) was constructed with samples in the age range 70-100, while that on the right (Young) was constructed with samples in the age range 20-60 to ensure that sufficient number of datapoints were available. Green- and purple-colored edges represent positive and negative correlations respectively. Thickness of the edges are directly proportional to the Spearman correlation value |r|. Blue and orange circles identify the classical and alternate butyrate producer species, respectively.

FIG. 17 shows the variation in alternate butyrate production pathways across age groups. Boxplots showing the relative abundance of alternative pathways for butyrate production in different age groups. FDR-adjusted p-values from a GLM test for age association are denoted by “*” (p-value≤0.05) and “***” (p-value<0.001). In all boxplots, the center line represents median, box limits represent upper and lower quartiles, and whiskers represent minimum and maximum values (outlier points are not included in the visualization).

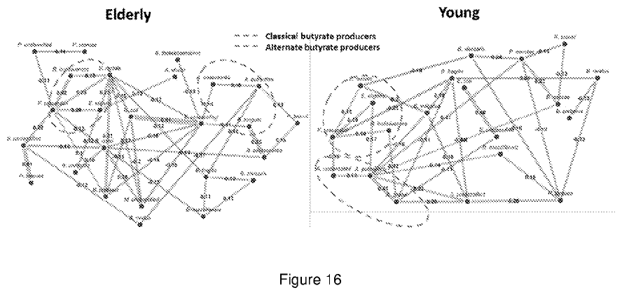

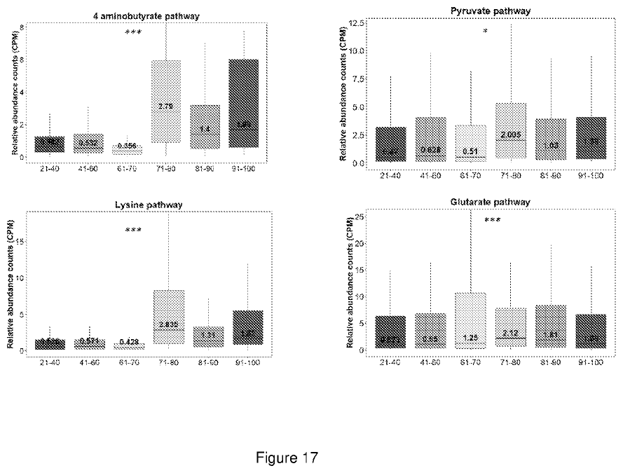

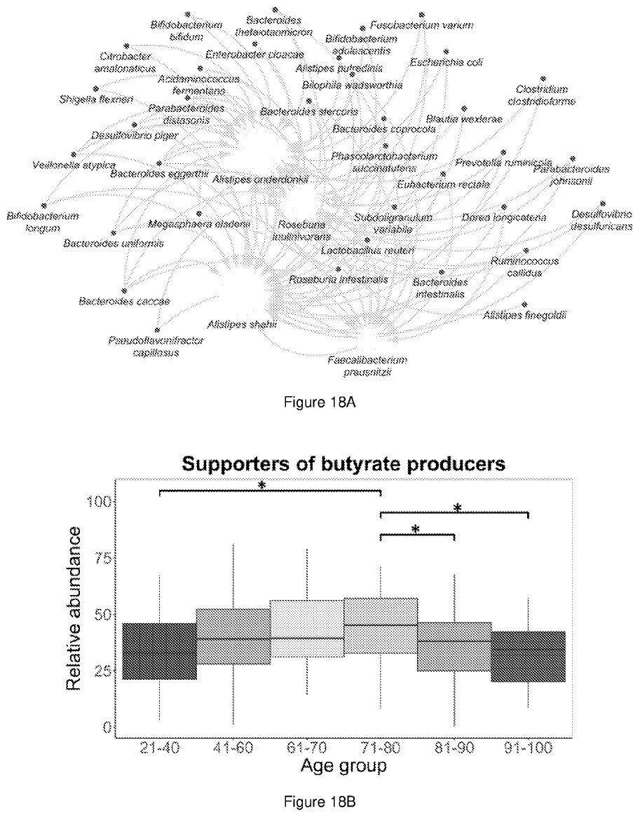

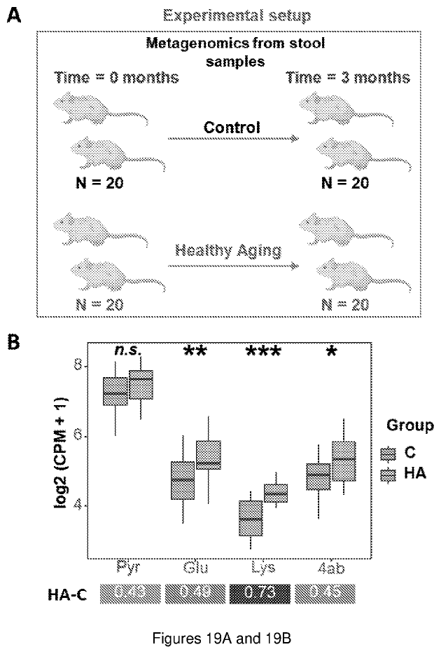

FIG. 18 shows the metabolic support in the gut microbiome for butyrate production. (A) Network figure depicting gut microbial species with high ‘metabolic support index’ for butyrate producers that receive maximum support (yellow nodes). Directed edges go from the supporting species to the one that is being supported, and the size of the supported node is proportional to the number of incoming edges. (B) Boxplots depicting combined relative abundances of all species that support butyrate producers (from subfigure A) in the gut microbiomes of subjects from various age groups. Wilcoxon test p-values<0.05 are indicated with the star symbol (‘*’). In all boxplots, the center line represents median, box limits represent upper and lower quartiles, and whiskers represent minimum and maximum values.

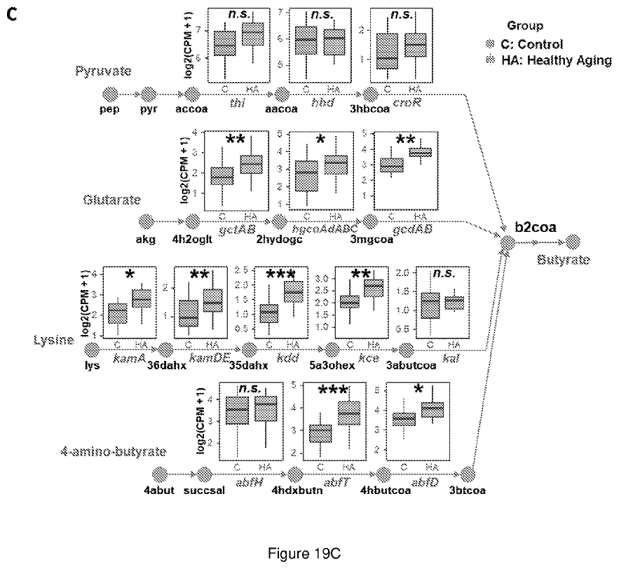

FIG. 19 shows the butyrate production pathways in a healthy aging mouse model. (A) Experimental setup for fecal metagenomic analysis in two groups of aged mice (18 months) with (Healthy Aging—HA) and without (Control—C) CaAKG 4% supplementation. (B) Boxplots showing relative abundance of alternative pathways for butyrate production in the two mouse groups after 3 months of supplementation. (C) Boxplots for abundance of various genes in corresponding pathways where nodes represent major metabolites and edge labels represent genes involved in their conversion. In all boxplots, center line represents median, box limits represent upper and lower quartiles, and whiskers represent minimum and maximum values. The labels “n.s.”. “*”. “**” and “***” represent FDR-adjusted p-value>0.05, p-value≤0.05, p-value<0.01 and p-value<0.001.

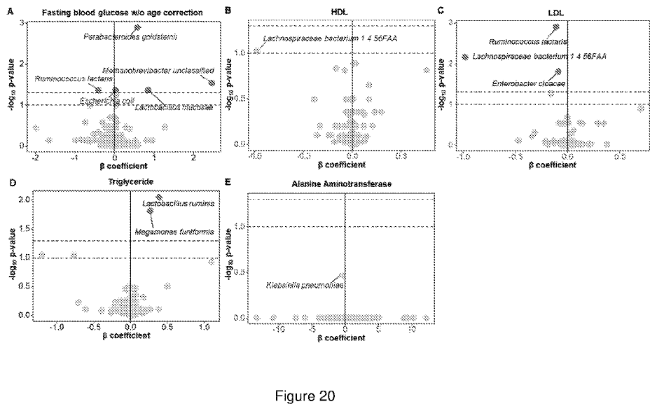

FIG. 20 shows the microbiome associations across various metabolic markers. Volcano plots showing β coefficient on the x-axis and FDR-adjusted −log₁₀ p-values (GLM test) on the y-axis. Red dots indicate p-value≤0.05 and grey dots indicate otherwise, with dotted lines marking p-value=0.05 and p-value=0.1 thresholds. (A) Fasting Blood Glucose: version of the analysis shown in FIG. 5 without including age as a covariate. (B, C, D) Results for HDL, LDL and Triglyceride levels. (E) Results for Alanine Aminotransferase showing a weak association with Klebsiella pneumoniae, compared to the significant association in FIG. 7 with AST.

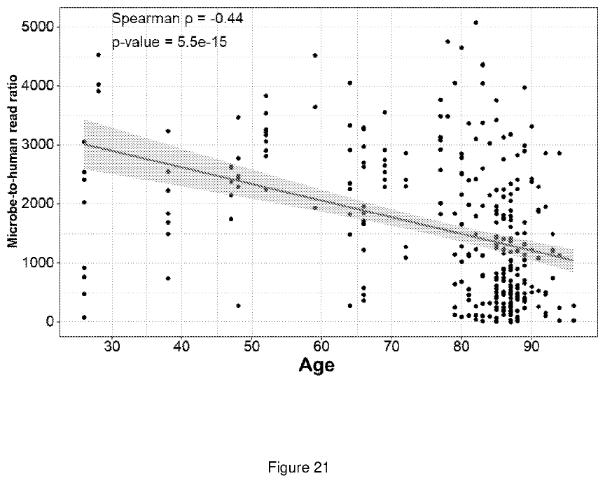

FIG. 21 shows the decrease in microbe-to-human read ratio with age. Scatterplot showing microbe-to-human read ratio (as a proxy for microbial biomass compared to human biomass) in relation to the age of subjects for the SG90 and CPE cohorts. Microbe-to-human read ratio was estimated as the proportion of microbial reads relative to human reads in the dataset (minimap2 mapping). Outlier points are not included in the visualization. Regression line (in blue) shows a negative correlation of gut microbial biomass with age.

Leveraging the availability of a well-phenotyped cohort of community-living octogenarians in Singapore, the present invention advantageously used deep shotgun metagenomic sequencing to do high-resolution taxonomic and functional characterization of gut microbiomes (n=234). Joint species-level analysis with other Asian cohorts identified a distinct age-associated shift in Asian gut metagenomes, characterized by a reduction in microbial richness, and enrichment of specific Alistipes species (e.g. Alistipes senegalensis, Alistipes onderdonkii, Alistipes shahii). Functional pathway analysis confirmed that these changes correspond to a metabolic switch in aging from microbial guilds that typically produce butyrate in the gut (e.g. Faecalibacterium prausnitzii, Roseburia inulinivorans) to alternate pathways that utilize amino-acid precursors. Extending these observations to key clinical markers helped identify >15 robust gut microbial associations to cardiometabolic health, inflammation, and frailty, including potential probiotics such as Parabacteroides goldsteinii and pathogenic species such as Dialister invisus, highlighting the role of the microbiome as biomarkers and potential intervention targets for promoting healthy aging.

The present invention will be described with respect to particular embodiments and with reference to certain drawings, but the invention is not limited thereto but only by the claims.

Any reference signs in the claims shall not be construed as limiting the scope. The drawings described are only schematic and are non-limiting. In the drawings, the size of some of the elements may be exaggerated and not drawn on scale for illustrative purposes. Where the term “comprising” is used in the present description and claims, it does not exclude other elements or steps. Where an indefinite or definite article is used when referring to a singular noun, e.g. “a”, this includes a plural of that noun unless something else is specifically stated. Furthermore, the terms first, second, third and the like in the description and in the claims, are used for distinguishing between similar elements and not necessarily for describing a sequential or chronological order. It is to be understood that the terms so used are interchangeable under appropriate circumstances and that the embodiments of the invention described herein are capable of operation in other sequences than described or illustrated herein.

The following terms or definitions are provided solely to aid in the understanding of the invention. Unless specifically defined herein, all terms used herein have the same meaning as they would to one skilled in the art of the present invention. The definitions provided herein should not be construed to have a scope less than understood by a person of ordinary skill in the art.

### EXAMPLE

In this invention, deep shotgun metagenomic analysis (n=234, >20 million reads on average) was used to study gut microbiomes in a cohort (SG90 cohort) of community-living octogenarians (primarily, age range=[71-100], see Table 4) in Singapore. The gut microbiomes of healthy octogenarians exhibited a defined shift in diversity and overall taxonomic composition, as a function of age and independent of other covariates. In addition, taxonomic analysis identified several species-level changes (e.g. enrichment of Alistipes senegalensis and Alistipes indistinctus), consistent with functional pathway analysis of the data, that points to a metabolic switch in aging from classical butyrate production to alternate pathways that utilize amino-acids (e.g. L-lysine) as precursor substrates. As butyrate derived from gut bacteria has diverse roles in host health (e.g. as energy for colonocytes, and reducing gut inflammation), we next associated gut microbiome composition with key markers for inflammation (e.g. CRP), cardiometabolic health (e.g. fasting blood glucose) and frailty (e.g. grip strength), to find additional microbial functions associated with healthy aging. Leveraging extensive clinical data and the size of the metagenomic datasets, >15 robust associations (accounting for demographic and clinical covariates that can be confounders) that highlight the role of the microbiome as biomarkers and potential intervention targets for promoting healthy aging were identified.

### Methods and Material

### 1. Cohorts and Datasets

SG90 cohort: The SG90 cohort is based on a longitudinal population health study that was set up in the 1990s which involved routine measurement of metabolic and other health variables. The current dataset is based on a subset of 234 elderly individuals (77-97 years old) who are community-living participants (not living in a nursing home, no diagnosis of dementia and not physically unfit) and consented to providing their stool and blood samples. The participants in the SG90 cohort were recruited under the SLAS-3 protocol approved by the Institutional Review Board (IRB) at National University of Singapore (reference number: B-15-081). This study was also approved by an IRB for the Singapore Chinese Health Study (reference number: H-17-027). Fasting blood glucose (mmol/L), triglyceride (mmol/L), total cholesterol (mmol/L), HDL (mmol/L), LDL (mmol/L), hs-CRP (mg/L), AST (U/L), ALT (U/L) and Vitamin B12 levels (pmol/L) were measured based on a blood draw collected either at the time of stool collection or within a week. In addition, physical assessments were performed to calculate BMI (kg/m²) from their weight (kg) and height (m), as well as measure gait speed (m/s) and handgrip strength (kg) of subjects.

SPMP dataset: The SPMP dataset is based on the recall of a subset of 109 healthy Singaporean subjects (53-74 years old) from a multi-omics study in Singapore. Stool samples were collected for gut microbiome analysis using shotgun metagenomic sequencing.

T2D dataset: Shotgun metagenomic datasets were obtained for 171 healthy Chinese individuals from a previously published type 2 diabetes (T2D) study using the curated MetagenomicData package. Briefly, the subjects chosen for our study were 21-70 years old and were non-diabetic controls in the study. Clinical data such as fasting blood glucose (mmol/L), triglyceride (mmol/L), total cholesterol (mmol/L), HDL (mmol/L) and LDL (mmol/L) levels were also obtained from this study.

CPE dataset: The CPE dataset is based on a prospective cohort study consisting of CPE-colonized subjects and their healthy family members. For our comparisons, we used shotgun metagenomic data of 82 healthy family members (21-80 years old) with Chinese ethnicity.

Demographic matching: Analysis for all cohorts was restricted to ethnic Chinese individuals and the gender balance across cohorts was found to be comparable (SG90: 59% female, T2D: 52% female, SPMP: 60% female, CPE: 60% female).

### 2. DNA Library Construction and Sequencing

PowerSoil DNA Isolation Kit (MO Bio Laboratories) was used for the extraction of DNA from stool samples. Minor modifications to the manufacturer's protocol were made (double the volume of C2, C3 and C4 buffers was added, and the duration of the centrifugation step was extended to twice the original duration). Purified DNA was eluted in 80 μL of C6 solution. DNA libraries were prepared using 50 ng of extracted DNA re-suspended in a volume of 50 μl. This was subjected to shearing using Adaptive Focused Acoustics™ (Covaris) with the following parameters-Duty Factor: 30%, Peak Incident Power (PIP): 450, 200 cycles per burst, Treatment Time: 240s. Sheared DNA was cleaned up with 1.5× Agencourt AMPure XP beads (A63882, Beckman Coulter) followed by end-repair, A-addition and adapter ligation using the Gene Read DNA Library 1 Core Kit (Qiagen) according to the manufacturer's protocol. Custom barcode adapters were used instead of GeneRead Adapter I Set for adapter ligation (see Table 3 below). Before enrichment, DNA libraries were cleaned twice using 1.5× Agencourt AMPure XP beads (A63882, Beckman Coulter) using the protocol from Multiplexing Sample Preparation Oligonucleotide kit (Illumina). Enrichment PCR was carried out with PE 1.0 and custom index-primers for 12 cycles. DNA Libraries were prepared with Agilent DNA1000 Kit (Agilent Technologies) by pooling equimolar concentrations and quantified using Agilent Bioanalyzer. DNA libraries were sequenced on an Illumina HiSeq X sequencing instrument generating >20 million 2×101 bp reads on average per library.

TABLE 3

| | |
|---|---|
| Barcode | 1^{st} strand: 5′P-GATCGGAAGAGCACACGTCT (SEQ ID NO: 1) |
| adapter, | 2^{nd} strand: 5′ACACTCTTTCCCTACACGACGCTCTTCCGATCT |
| double | (SEQ ID NO: 2) |
| stranded | |
| PE 1.0 | 5′AATGATACGGCGACCACCGAGATCTACACTCTTTCCCTACACGACGCTC |
| | TTCCGATC* T (SEQ ID NO: 3) |
| Index | 5′CAAGCAGAAGACGGCATACGAGATXXXXXXXXGTGACTGGAGTTCAGAC |
| Primer | GTGTGCTCTTCCGATC*T (SEQ ID NO: 4) |

### 3. Read Pre-Processing and Profiling

Illumina shotgun metagenomic sequencing reads were processed using a Nextflow pipeline (https://github.com/CSB5/shotgunmetagenomics-nf). Briefly, raw reads were filtered to remove low quality bases and adapter sequences were removed using fastp (v0.20.0) with default parameters. Human reads were removed by mapping to the hg19 reference using BWA-MEM (v0.7.17-r1188, default parameters) and samtools (v1.7). The remaining reads were used for taxonomic profiling using MetaPhlAn2 (v2.7.7, default parameters). Functional profiles for the metagenomes were obtained using HUMAnN2 (v2.8.1). For all statistical tests, Benjamin Hochberg's false discovery rate method was used to correct for multiple testing at a significance threshold of 5%.

4. Statistical Analysis with Taxonomic Profiles

Taxonomic profiles were corrected for batch effects with MMuphin using age group as a covariate. MMUPHin is a batch correction method that employs an empirical Bayes approach to model read counts with respect to batch variables and biologically relevant covariates (age group in our case). It then gives as output batch-corrected count data which aims to retain the effects of biologically relevant covariates. We conducted a sanity-check on batch corrected profiles to confirm that cohort effects were reduced. Species level relative abundances (>0.1%) were then used to compute alpha diversity indices (Shannon and Simpson), Pielou's evenness and the beta diversity index (Bray-Curtis distance) using the R package vegan and visualized using ggplot2. Robustness of computed metrics to variations in sequencing depth was confirmed based on sub-sampling and correlation analysis. Ordinal logistic regression in R was used to test for statistical significance in relation to different age groups. A generalized linear model (GLM) approach, as commonly used in genetic association studies, was used to test associations between the relative abundance for each species (SA) and age as a continuous variable, with covariates for gender (G), body mass index (BMI), fasting blood glucose (FBG), triglycerides (TGL), total cholesterol (TC), high density lipoprotein (HDL) and low-density lipoprotein (LDL) levels (i.e. with the formula: Age ˜SA+G+BMI+FBG+TGL+TC+HDL+LDL). Only taxa with >50 non-zero values were tested to avoid spurious associations. Missing clinical covariates were imputed through an Expectation Maximization algorithm.

To assess reproducibility of taxonomic associations, three distinct approaches were used: (i) GLM analysis using all four cohorts with batch correction as described above, (ii) GLM analysis using the original data for the SG90 and CPE cohorts as they were similarly processed, and (iii) Trend analysis with the Cochran-Armitage test after conversion of relative abundance data into presence-absence values (cutoff of 0.1%). Microbial associations in same direction found in two out of the three methods were considered reproducible with the reported p-values being the median across all methods. In order to further test the robustness of associations, parameters and options were varied for all three approaches including normalization technique (Total Sum Scaling-TSS and Cumulative Sum Scaling-CSS), association analysis technique (ANCOM-BC, MaAsLin2) and relative abundance cutoffs (0.05%-1%; see FIG. 11). CCREPE was used to compute Spearman correlation values and identify bacterial species with strong co-occurrence patterns in different age groups (p>0.2, p-value<0.05).

### 5. Pathway and Gene-Level Analysis

The HMP Unified Metabolic Analysis Network (HUMAnN2) pipeline was used to determine the relative abundance of microbial pathways in different gut metagenomes. The default Kyoto encyclopedia of genes and genomes (KEGG) catalog was used as the pathway reference. Unstratified relative abundance values for SG90 and SPMP were integrated with HUMAnN2 results for T2D from curated Metagenomeseq for shared pathways, and significant differentially abundant pathways across age groups were determined based on linear discriminant analysis with IEfSe (p-value<0.05 and LDA score>3).

For gene-level analysis of metabolic pathways involved in butyrate production, EC numbers of enzymes were mapped to corresponding UniRef IDs from the HUMAN2 output. For every gene, read counts were obtained by taking the sum for all corresponding UniRef values and statistical analyses were performed using R with visualizations from ggplot2.

### 6. Mouse Experiments

Two independent groups of 18 month old C57BL/6 mice (n=20 each, regular chow diet) were housed separately (maximum of four mice per cage). Mice in the control group were on the regular chow diet while those in the healthy aging group were switched to a diet containing 4% calcium alpha-ketoglutarate, with stool samples collected at baseline and 3 months after diet switch for metagenomic analysis. Genomic DNA was extracted from mouse stools using QIAamp PowerFecal Pro DNA Kit (Qiagen), according to manufacturer's instructions. DNA was quantified on a Qubit Fluorometer using the Qubit dsDNA BR Assay Kit (ThermoFisher Scientific). Purified genomic DNA (50 ng) was used for library construction steps using NEBNext® Ultra™ II FS DNA Library Prep Kit according to manufacturer's instructions. Finally, each library sample was assessed for quality based on fragment size and concentration using the Agilent D1000 ScreenTape system, with samples adjusted to identical concentrations by means of dilution and volume-adjusted pooling. The multiplexed sample pool was paired-end (2×151 bp) sequenced on an Illumina HiSeq X Ten system. Mouse gut metagenomes were annotated using eggNOG-mapper¹¹⁶ (v2.1.9). Briefly, reads were mapped against eggNOG protein database (eggnog 5.0¹¹⁷) using DIAMOND¹¹⁸ in blastx mode. The default KEGG¹¹⁴ catalog was used as the pathway reference. For gene-level analysis of metabolic pathways involved in butyrate production, the corresponding KEGG IDs of each gene were used from the eggNOG-mapper's output. For every gene, read counts were obtained by taking the sum for all corresponding KEGG values. Gene normalized abundances were measured as logarithm of counts per millions [log (CPM+1)] to adjust for differences in sequencing depths. Pathway normalized abundances were calculated by taking the sum of all of gene counts for each pathway and log-transformed and normalized to sequencing depth as log (CPM+1). Statistical tests for groups comparisons were done using Wilcoxon rank-sum test (FDR adjusted p-value<0.05 considered to be significant) and were performed using R with visualizations created using ggplot2.

### 7. Phenotypic Association Analysis

Associations of microbial species with clinical phenotypic markers were identified based on linear regression using ‘glm’ function in R, with age and other markers serving as covariates for adjustment. Only species present in at least ten samples were considered for this analysis. Phenotypic markers available in two datasets (T2D and SG90) included body mass index (BMI), fasting blood glucose, triglycerides, total cholesterol, high-density lipoprotein (HDL) and low-density lipoprotein (LDL) levels. For hs-CRP, AST, and ALT levels, gait speed, right handgrip strength, left handgrip strength, sleep duration and free thyroxine levels, the associations were only tested for SG90 samples (without batch-correction) where this data was available.

### 8. Metabolic Network Analysis

Metabolic support index (MSI) values for the species identified in Tables 1A and 1B in relation to other gut microbial species were obtained based on metabolic network analysis, as described previously. Briefly, MSI uses network flow analysis to quantify the extent of microbial metabolism that is enabled by the presence of other species in the community. The metabolic support network was visualized as a directed graph using Cytoscape (v3.8.0). Species that are known butyrate producers and receive the most metabolic support (based on in-degree of nodes) were highlighted in the network. The combined relative abundances of organisms supporting these butyrate producers were compared across age groups using the Wilcoxon test.

### Results and Discussion

### 1. Shotgun Metagenomics Reveals a Defined Aging-Associated Shift in Microbial Richness and Functional Guilds

To study the role of the gut microbiome in healthy aging, stool samples from a deeply phenotyped cohort of community-living Asian octogenarians (SG90, see Table 4) were analyzed with deep shotgun metagenomic sequencing (n=234, 27 million reads on average).

TABLE 4
Clinical characteristics of the SG90 cohort.

| | | |
|---|---|---|
| All Subjects | Male | Female |

Demographics

| | | | |
|---|---|---|---|
| Number of subjects | 234 | 95 | 139 |
| Age (Mean ± SD) | 86 ± 4 | 86 ± 3 | 86 ± 4 |

Serum measurements (Mean ± SD)

| | | | |
|---|---|---|---|
| High-sensitivity CRP (mg/L) | 3.7 ± 8.4 | 4.6 ± 11.9 | 3.1 ± 4.4 |
| Fasting blood glucose (mmol/L) | 5.2 ± 0.4 | 5.3 ± 0.5 | 5.2 ± 0.4 |
| Triglycerides (mmol/L) | 1.4 ± 0.6 | 1.2 ± 0.6 | 1.5 ± 0.7 |
| Total cholesterol (mmol/L) | 4.6 ± 1.1 | 4.4 ± 1.0 | 4.8 ± 1.1 |
| High-density lipoprotein (mmol/L) | 1.4 ± 0.3 | 1.3 ± 0.3 | 1.4 ± 0.4 |
| Low-density lipoprotein (mmol/L) | 2.6 ± 0.9 | 2.5 ± 0.8 | 2.7 ± 0.9 |
| Alanine aminotransferase (U/L) | 18.4 ± 8.0 | 19.3 ± 8.1 | 17.8 ± 8.0 |
| Aspartate aminotransferase (U/L) | 25.0 ± 6.8 | 25.5 ± 7.6 | 24.6 ± 6.2 |

Disease incidence (%)

| | | | |
|---|---|---|---|
| Type 2 diabetes | 18 | 12 | 22 |
| Heart attack | 6 | 9 | 5 |
| Heart failure | 4 | 4 | 4 |
| Cataracts/glaucoma | 36 | 31 | 38 |
| Kidney failure | 0.9 | 1 | 0.7 |
| Asthma | 3 | 1 | 4 |
| COPD | 2 | 4 | 0.7 |
| Arthritis | 9 | 5 | 12 |
| Osteoporosis | 4 | 1 | 6 |
| Hip fracture | 4 | 1 | 6 |
| Parkinson's disease | 0.9 | 2 | 0 |
| Other neurodegenerative disorders | 0.4 | 1 | 0 |
| Gastrointestinal problems | 6 | 5 | 7 |
| Thyroid problems | 2 | 3 | 1 |
| Cancer | 3 | 2 | 3 |

The resulting high-resolution species-level taxonomic profiles were compared to reference shotgun metagenomic data from healthy, younger Singaporeans from two cohorts (SPMP, n=109, age range=[53-74]; CPE, n=96, age range=[21-80]), as well as other Asian populations (T2D, n=171, age range=[21-70]). Systematic joint analysis of these cohorts after batch-correction and matching other demographic characteristics (gender, ethnicity), revealed a progressive shift in taxonomic profiles across different age groups (n=516, age range=[21-100], see FIG. 1B), particularly along the y=x axis relative to the first and second principal components of variation (ordinal logistic test p-value<0.001, see FIGS. 1C and 12A). Diversity analysis indicated that this was accompanied by an aging-associated shift (ordinal logistic test p-value<0.001, see FIG. 1D) that is driven by microbial richness (see FIG. 12B) and evenness, both of which exhibit significant reduction with age (see FIGS. 12C and D). PERMANOVA analysis showed that Age was the major source of variation compared to other attributes (see FIG. 13A) and that beta diversity peaked first in the 41-60 age group and then again in the 91-100 age group (see FIG. 13B), consistent with prior observations on higher diversity in extreme aging groups. These results highlight an aging-associated shift in microbial composition that is defined by the loss of gut species, and increasing uniqueness in microbiome composition in extreme age groups.

In order to identify taxa associated with age, a generalized linear model (GLM) was used to account for demographic and clinical covariates that can be confounders (e.g. gender, body mass index, fasting blood glucose, triglyceride, total cholesterol, high density lipoprotein and low-density lipoprotein levels). Overall, 4 phyla, 20 genera and 43 species were found to be associated significantly with age (FDR-adjusted p-value<0.05, see Tables 2A and 2B, and Tables 7A and 7B).

TABLE 7A
microorganisms enriched with healthy aging

| | FDR- | | | | |
|---|---|---|---|---|---|
| | adjusted | β | Standard | | |
| Species | p-value | estimate | Error | 2.50% | 97.50% |
| Alistipes senegalensis | 7.77 × 10⁻⁵ | 19.61 | 4.43 | 10.92 | 28.30 |
| Alistipes indistinctus | 0.01 | 3.21 | 1.09 | 1.07 | 5.34 |
| Alistipes unclassified | 5.97 × 10⁻⁵ | 2.48 | 0.55 | 1.41 | 3.54 |
| Bacteroides xylanisolvens | 0.006 | 2.46 | 0.65 | 1.20 | 3.73 |
| Bacteroidales bacterium ph8 | 5.97 × 10⁻⁵ | 1.61 | 0.36 | 0.91 | 2.30 |
| Alistipes shahii | 0.001 | 1.59 | 0.29 | 1.03 | 2.15 |
| Bacteroides clarus | 0.049 | 1.37 | 0.58 | 0.24 | 2.50 |
| Klebsiella pneumoniae | 0.01 | 1.14 | 0.39 | 0.38 | 1.90 |
| Escherichia unclassified | 2.16 × 10⁻⁵ | 1.09 | 0.22 | 0.65 | 1.52 |
| Parabacteroides merdae | 0.03 | 0.95 | 0.50 | −0.03 | 1.93 |
| Parabacteroides distasonis | 0.04 | 0.83 | 0.35 | 0.15 | 1.51 |
| Bacteroides dorei | 6.46 × 10⁻⁵ | 0.78 | 0.14 | 0.51 | 1.04 |
| Alistipes onderdonkii | 0.002 | 0.66 | 0.19 | 0.29 | 1.03 |
| Escherichia coli | 0.045 | 0.26 | 0.11 | 0.04 | 0.48 |

TABLE 7B
microorganisms depleted in healthy aging

| | FDR-adjusted | β | Standard | | |
|---|---|---|---|---|---|
| Species | p-value | estimate | Error | 2.50% | 97.50% |
| Coprococcus catus | 0.00011 | −25.41 | 5.88 | −36.93 | −13.89 |
| Parasutterella | 0.00037 | −19.26 | 4.81 | −28.68 | −9.84 |
| excrementihominis | | | | | |
| Dorea formicigenerans | 1.83 × 10⁻⁷ | −15.26 | 2.61 | −20.37 | −10.15 |
| Coprococcus comes | 1.36 × 10⁻⁸ | −11.48 | 1.80 | −15.01 | −7.95 |
| Ruminococcus obeum | 5.12 × 10⁻¹⁴ | −11.08 | 1.32 | −13.67 | −8.49 |
| Megamonas funiformis | 0.01034 | −9.31 | 3.15 | −15.48 | −3.14 |
| Eubacterium hallii | 1.08 × 10⁻¹³ | −8.93 | 1.44 | −11.75 | −6.10 |
| Lachnospiraceae | 0.00037 | −7.90 | 1.96 | −11.74 | −4.05 |
| bacterium 5 1 63FAA | | | | | |
| Clostridium leptum | 0.00198 | −6.47 | 3.60 | −13.53 | 0.58 |
| Ruminococcus lactaris | 7.83 × 10⁻⁵ | −6.44 | 1.46 | −9.31 | −3.58 |
| Bilophila unclassified | 6.38 × 10⁻⁵ | −6.34 | 1.64 | −9.55 | −3.13 |
| Eubacterium ventriosum | 5.97 × 10⁻⁵ | −6.00 | 1.33 | −8.60 | −3.39 |
| Dorea longicatena | 1.39 × 10⁻¹⁰ | −5.27 | 1.13 | −7.48 | −3.06 |
| Ruminococcus callidus | 0.00552 | −4.66 | 1.46 | −7.52 | −1.80 |
| Lachnospiraceae | 7.34 × 10⁻⁵ | −4.48 | 1.01 | −6.45 | −2.51 |
| bacterium 1 1 57FAA | | | | | |
| Roseburia inulinivorans | 2.17 × 10⁻⁵ | −2.33 | 0.48 | −3.27 | −1.38 |
| Bifidobacterium | 6.72 × 10⁻⁶ | −1.76 | 0.49 | −2.72 | −0.80 |
| pseudocatenulatum | | | | | |
| Roseburia intestinalis | 0.01034 | −1.49 | 0.50 | −2.48 | −0.50 |
| Eggerthella unclassified | 0.00911 | −1.47 | 1.93 | −5.26 | 2.32 |
| Ruminococcus sp 5 1 | 5.72 × 10⁻¹⁸ | −1.46 | 0.45 | −2.34 | −0.58 |
| 39BFAA | | | | | |
| Eubacterium eligens | 0.03193 | −1.33 | 0.53 | −2.36 | −0.29 |
| Ruminococcus torques | 0.00633 | −1.27 | 0.40 | −2.06 | −0.48 |
| Faecalibacterium | 4.08 × 10⁻⁷ | −1.08 | 0.15 | −1.37 | −0.79 |
| prausnitzii | | | | | |
| Megamonas | 0.03193 | −0.90 | 0.36 | −1.60 | −0.20 |
| unclassified | | | | | |
| Ruminococcus gnavus | 0.00152 | −0.79 | 0.33 | −1.43 | −0.15 |
| Eubacterium rectale | 0.00635 | −0.75 | 0.16 | −1.06 | −0.44 |
| Ruminococcus bromii | 0.03402 | −0.63 | 0.25 | −1.13 | −0.13 |
| Streptococcus salivarius | 0.0474 | −0.51 | 0.42 | −1.33 | 0.31 |
| Bacteroides massiliensis | 0.02519 | −0.47 | 0.20 | −0.86 | −0.07 |

Tables 7A and 7B are an expanded list of Tables 1A and 1B

The phylum and genus level results were broadly consistent with prior observations (e.g. in Chinese, Japanese and Italian centenarians), with enrichment in Bacteroidetes (β=0.34, FDR-adjusted p-value<0.001), and depletion in Firmicutes (β=−0.50, FDR-adjusted p-value<0.001) with age (see Tables 2A and 2B). At the genus level a strong enrichment in unclassified Acidaminococcaceae species (β=7.70, FDR-adjusted p-value=0.013) and depletion in 10 Parasutterella species (B=−20.47, FDR-adjusted p-value<0.001) was observed that has not been noted before, while at the species level a majority of the observations were novel owing to the higher resolution of shotgun-metagenomics (see Tables 1A and 1B, and 7A and 7B).

In addition, the robustness of this analysis was tested by varying the normalization approach, statistical model and cohorts used to find that most taxa associations were detected in a majority of the conditions tested (see FIG. 11). It was also noted that while several species associations were detected in individual cohorts (n=13), the inclusion of the SG90 cohort notably boosted the number of age-associations identified (n=37), suggesting that having a wider age range and more subjects could benefit such analysis (see FIG. 14).

Among the core gut microbiome taxa identified, many species in the Bacteroides genus showed significant positive and negative associations including Bacteroides dorei, Bacteroides xylanisolvens and Bacteroides massiliensis (Tables 1A and 1B, FIGS. 1E, 2B), though this variation can be masked at the genus level highlighting the utility of species-level resolution offered by shotgun metagenomics. It was also noted an enrichment with age for multiple Alistipes species, including A. shahii, A. onderdonkii and A. senegalensis, that are bile tolerant, have a unique way of fermenting amino acids, and can produce neurotransmitter precursors such as indole (see FIG. 15A). In contrast, several species that are known to be important for butyrate production in the gut, including Faecalibacterium prausnitzii, Roseburia inulinivorans and Eubacterium rectale, were significantly depleted with age (FIG. 2, see FIG. 15B). Taxonomic co-occurrence analysis highlighted a distinct network structure in the elderly compared to younger individuals (fewer edges and hubs), with a shared pattern of cliques formed by the classical butyrate producers and Alistipes species, indicating that these may represent distinct functional guilds that switch roles with age (see FIG. 16).

TABLE 2A

| Enriched with age | GLM p-value | β for age [95% C.I.] | |
|---|---|---|---|
| Bacteroidetes | 9.28E−11 | 0.34 | [0.27, 0.41] |
| Depleted with age | | | |
| Firmicutes | 3.70E−29 | −0.50 | [−0.59, −0.43] |
| Actinobacteria | 1.79E−07 | −0.47 | [−0.63, −0.30] |
| Fusobacteria | 0.0087 | −4.00 | [−6.76, −1.22] |

TABLE 2B

| Enriched with age | GLM p-value | β for age [95% C.I.] | |
|---|---|---|---|
| Bacteroidales noname | 8.95E−06 | 1.49 | [0.90, 2.09] |
| Alistipes | 1.98E−04 | 0.38 | [0.21, 0.54] |
| Bacteroides | 6.80E−04 | 0.18 | [0.095, 0.26] |
| Acidaminococcaceae | 0.013 | 7.70 | [2.39, 13.01] |
| unclassified | | | |
| Klebsiella | 0.017 | 0.92 | [0.26, 1.57] |
| Escherichia | 0.019 | 0.21 | [0.057, 0.37] |
| Depleted with age | | | |
| Dorea | 2.26E−19 | −7.16 | [−8.94, −5.38] |
| Roseburia | 2.11E−07 | −1.74 | [−2.30, −1.18] |
| Faecalibacterium | 2.17E−07 | −1.12 | [−1.40, −0.84] |
| Ruminococcus | 6.18E−07 | −1.05 | [−1.44, −0.67] |
| Blautia | 1.18E−06 | −1.16 | [−1.59, −0.74] |
| Bilophila | 2.25E−06 | −7.60 | [−10.45, −4.74] |
| Bifidobacterium | 5.16E−05 | −0.41 | [−0.59, −0.23] |
| Parasutterella | 0.00010 | −20.47 | [−29.86, −11.08] |
| Eubacterium | 0.00013 | −0.60 | [−0.89, −0.30] |
| Coprococcus | 0.00096 | −2.92 | [−4.48, −1.36] |
| Eggerthella | 0.0014 | −1.88 | [−5.18, 1.41] |
| Lachnospiraceae noname | 0.0019 | −2.96 | [−4.67, −1.24] |
| Acidaminococcus | 0.0027 | −0.22 | [−0.91, 0.47] |
| Fusobacterium | 0.018 | −3.71 | [−6.40, −1.02] |

Tables 2A and 2B—Higher-level taxonomic associations with age. At the (A) phylum and (B) genus level, the table reports taxa that are statistically significant in at least 2 out of 3 primary analysis approaches (see Methods). The p-values reported here are the median FDR-adjusted p-values from these 3 approaches. The β coefficient estimates strength of association as change in age (in years) for every 1% increment of taxa relative abundance.

As such, 42 microbial species in the gut microbiome were identified to exhibit a robust association with aging independent from other disease risk phenotypes. These species catalyse a switch in the gut microbial metabolic capacity to maintain butyrate production during healthy ageing. Similarly, the effect of age was untangled to identify independent sets of microbiome markers that have robust associations with multiple disease phenotypes. These species can serve as potential indicators to identify various disease phenotypes.

The shotgun metagenomic samples of gut microbiome of 434 samples were systematically analyzed with a wide range of age between 21-100 years that have health data collected for disease risk factor analyses. The shift in taxonomic composition with age was demonstrated, see FIG. 1A.

To identify robust microbial taxa associating independently with age, regression analysis was used to model age with covariates adjustment of the disease risk phenotypes to increase the sensitivity and specificity of the association analysis. In total, we identified 43 bacterial species to independently associate with age significantly. The 7 common significant species found in at least 50% of the samples that are enriched with age include Alistipes onderdonkii, Alistipes putredinis, Bacteroides caccae, Bacteroides xylanisolvens, Escherichia coli, Escherichia unclassified and Subdoligranulum unclassified. Species that are depleted include Faecalibacterium prausnitzii, Roseburia inulinivorans, Eubacterium rectale, Bacteroides vulgatus, Bacteroides stercoris, Bilophila unclassified and Parabacteroides merdae. The enriched versus depleted species for ageing had a switch in functionality for butyrate production, see FIG. 2.

### 2. Gut Microbiomes in Elderly Asians Exhibit a Metabolic Switch in Butyrate Synthesis Pathways

To further analyze metabolic changes associated with aging, shotgun metagenomic data from all subjects was used to quantify gene and pathway abundances. In total, 413 metabolic pathways were identified and quantified of which 74 were found to be differentially abundant across various age groups, broadly grouping into the four categories of sugar metabolism (13 pathways), vitamin and energy metabolism (30 pathways), lipid metabolism (12 pathways) and amino acid metabolism (19 pathways, FIG. 3). Specifically, among sugar metabolism pathways, distinct age-associated biases were observed, where e.g., pyruvate fermentation to acetate and lactate was enriched in the younger age group (21-40), while degradation of mono-, di- and polysaccharides was enriched in older age groups (FIG. 3A). As many as 8 different pathways for various substrates were enriched in the 61-70 age group, including simple pentose sugars (Pentose phosphate pathways), and complex polysaccharides such as stachyose and starch (Starch degradation V). Notably, microbial glycolytic pathways for metabolism of simple sugars (glucose) were enriched in healthy individuals over 90, while prior work had highlighted the role of corresponding pathways in aging in model organisms.

In line with the vital role of vitamins as essential micronutrients (particularly in energy metabolism and immune function) with limited synthesis in humans, several microbial pathways were associated with aging (especially B vitamins, FIG. 3B). In particular, thiamine diphosphate and flavin biosynthesis were enriched in the most elderly (91-100) and could have anti-inflammatory and anti-aging roles. Similarly, four biosynthetic pathways producing phospho-pantothenate and thiamine were abundant in elderly groups (71-80 and 61-70), while alternate pathways to produce biotin and pantothenate were enriched in younger individuals (21-40).

While microbial lipid metabolism pathways were predominant in younger subjects (21-40 age group, FIG. 3C), pathways related to amino acid metabolism were more often enriched in the elderly (particular 61-70 age group), including the synthesis of various essential branched-chain and aromatic amino acids such as histidine, valine, threonine, isoleucine, tryptophan, and methionine (FIG. 3D). In addition, lysine biosynthesis was over-represented in the gut microbiomes of two older age groups (61-70 and 81-90), linking to the earlier observation of age-related enrichment in specific Alistipes species which are known to produce short-chain fatty acids such as butyrate with lysine as substrate. Analysis of the four major known pathways for butyrate production (from pyruvate, glutarate, 4-amino butyrate and lysine) confirmed that while several lysine-associated genes show an age-related increasing trend (e.g. kamA, FDR-adjusted p-value<0.01 and kdd, FDR-adjusted p-value<0.05), no significant associations were seen in the pyruvate related genes (FIG. 4). Furthermore, the overall gene abundance in the pathway for butyrate production from lysine also showed a significant increase with age (FDR-adjusted p-value<0.001, see FIG. 17). Metabolic pathways analysis revealed that the abundance of gut microbial species that provide metabolic support to butyrate producers peaked in older age groups (71-80; see FIG. 18). These observations, along with those in the previous section, indicate a metabolic switch in butyrate synthesis pathways from pyruvate to lysine in the gut microbiomes of elderly Asians.

To further explore these findings, a healthy aging model in mice was used, where dietary supplementation with alpha ketoglutarate (AKG) to aged mice (18 months) not only increased lifespan but also improved inflammation and frailty markers. Shotgun metagenomics of fecal samples from AKG-supplemented and control mice (n=20 per group, see FIG. 19A) showed that even though mice harbor distinct gut microbial species, at the pathway level a similar shift in butyrate production pathways is seen in healthy aging mice (see FIG. 19B). In particular, the lysine to butyrate conversion pathways shows the strongest enrichment, as seen in the human data (1.65-fold increase in median abundance, FDR-adjusted p-value<0.01). At the gene level, several key genes for lysine to butyrate production were strongly enriched including kdd, kamDE, kce and kamA (FDR-adjusted p-value<0.05; see FIG. 19C). Such a trend was not observed for genes involved in pyruvate to butyrate conversion, highlighting the role of a switch in butyrate synthesis pathways to support healthy aging.

Congruously in the analysis on microbial gene function, the enrichment of specific metabolic pathways with ageing was revealed and point towards the conversion of L-lysine to butyrate. 74 gene pathways were found to be significantly enriched in various age groups. These pathways were grouped into four broad categories viz, Sugar metabolism (13 pathways), Vitamin and Energy metabolism (30 pathways), Lipid metabolism (12 pathways) and amino acid metabolism (19 pathways), see FIG. 3.

Alistipes sp. are known to produce short-chain fatty acids such as butyrate with lysine as substrate. Butyrate production in the gut happens via four major pathways that originate from pyruvate, glutarate, 4-amino butyrate and lysine. It was found here that several lysine-associated pathways and the genes to show an age-related increasing trend, indicating that these play a role in healthy aging. Here, it is shown in FIG. 4 that an enrichment in the conversion of L-lysine to SCFA (butyrate) and not the pathways converting central carbon metabolites to SCFAs.

The enrichment in L-lysine biosynthesis in the subjects in the age group of 61-70 and 91-100 was observed, see FIG. 3D. L-lysine can also be converted to short chain fatty acids, particularly butyrate by species belonging to Alistipes genera. From the observations from the taxa analysis where an increased abundance of Alistipes sp in older age groups was noted, it was hypothesized that there is an age-based switch in the metabolic pathway producing butyrate. Further confirming this, the analysis focusing on the genes involved in various metabolic pathways producing butyrate displayed a significant enrichment of genes involved in lysine to butyrate pathway, see FIG. 4.

Robust species-level associations between gut microbial taxa and aging phenotypes

Aging is a significant risk factor for chronic diseases impacting multiple organ functions, including metabolic, immune, and musculoskeletal systems, which could be mediated via gut microbiome function.

To further relate the taxonomic and functional changes observed in Asian octogenarians with their clinical phenotypes, the higher resolution of shotgun metagenomic data, broader age range and size of our cohorts were harnessed, to robustly identify associations in a generalized linear model framework while accounting for demographic and clinical confounders (e.g. age, gender, other metabolic markers. Despite the multiplicity of taxa and clinical features, we noted that this analysis typically resulted in a few significant associations (FIGS. 5 to 10). For example, for fasting blood glucose as a well-studied biomarker for glucose metabolism, insulin resistance, and type 2 diabetes, one associated species was identified: Parabacteroides goldsteinii (β=0.49, p-value<0.01, FIG. 5), with prior in vivo observations supporting this link. Note that β values indicate the change in response variable (e.g. fasting blood glucose levels) for every unit change in relative abundance values, after accounting for other confounding variables. Given the known normal range of fasting blood glucose levels (<6.1 mmol/L; see Table 5) the observed β (0.49) therefore suggests a large effect size. Furthermore, we observed a previously reported association for E. coli with fasting blood glucose, although this was not statistically significant after accounting for age (see FIG. 20A).

TABLE 5
Reference ranges for various clinical phenotypes.

| Clinical phenotypes | Range | |
|---|---|---|
| HDL | 1.0-1.5 | mmol/L |
| LDL | 2.6-3.3 | mmol/L |
| Triglyceride | 1.7-2.2 | mmol/L |
| Total cholesterol | <5.2 | mmol/L |
| Fasting blood glucose | <6.1 | mmol/L |
| High-sensitivity CRP | <1 | mg/L |
| Vitamin B12 | 160 to 950 | pmol/L |
| Alanine Aminotransferase | 6-66 | U/L |
| Aspartate | 6-35 | U/L |
| Aminotransferase | | |

Healthy ranges for various clinical phenotypes that were analyzed in this study.

It was also highlighted the beneficial roles of Lachnospiraceae 1 4 56FAA in maintaining healthy levels of good cholesterol and lowering the bad cholesterol. Since total cholesterol is a sum of LDL+HDL, Lachnospiraceae 1 4 56FAA helps in maintaining higher levels of good cholesterol (HDL) by keeping LDL to minimum. Cholesterol biomarkers (e.g. high-density lipoproteins-HDL, low-density lipoproteins-LDL and triglycerides) for hypercholesterolemia and cardiovascular diseases were also analyzed for their impact on healthy aging. Association analysis with total cholesterol levels revealed two significant candidates, positive association with abundance of Lachnospiraceae bacterium 1 4 56FAA (B=1.16, FDR-adjusted p-value=5.16×10⁴) and Ruminococcus lactis (B=0.1, FDR-adjusted p-value=0.02, FIG. 6), neither of which has been reported previously 58. Interestingly, analysis with LDL levels revealed the opposite trend, with the Lachnospiraceae species (B=−0.98, FDR-adjusted p-value=1.21×10⁻³) and R. lactis (B=−0.11, FDR-adjusted p-value=6.74×10⁻³) being negatively correlated, and similar trends being observed with HDL but not reaching statistical significance (see FIGS. 20B and 20C). These observations indicate potential beneficial roles of Lachnospiraceae species in maintaining lower levels of LDL. Interestingly, microbial associations with triglyceride levels were dominated by positive associations with Megamonas funiformis, a species that has been described to be strongly enriched in post-cholecystectomy patients and individuals with increased risk of cardiovascular disease, and Lactobacillus ruminis whose enrichment has been reported in ischemic stroke patients (see FIG. 20D).

As the biological processes of aging are in part believed to be accelerated by inflammation (inflammaging), including risk for cardiovascular disease, frailty and disability, the identification of gut microbial associations with inflammation markers was sought. Specifically, serum levels of aspartate aminotransferase (AST) were associated with the abundance of Klebsiella pneumoniae (B=0.65, FDR-adjusted p-value=0.01, FIG. 8), a sporadic colonizer of the gut microbiome, and a known pathogen for liver abscesses that are increasingly common in Asian populations.

A similar association was seen with alanine aminotransferase (ALT), a more specific biomarker for liver injury, but it did not reach statistical significance (see FIG. 20E). Analysis with high-sensitivity C-reactive protein (hs-CRP) levels, a commonly used marker for systemic inflammation, identified a known association with Escherichia coli (β=0.20, FDR-adjusted p-value=0.04), as well as associations with Streptococcus species (S. infantarius: B=20.25, FDR-adjusted p-value=0.01, and S. salivarius: β=1.99, FDR-adjusted p-value=0.03) which have been linked with rheumatoid arthritis (FIG. 7). Furthermore, significant associations were observed with an Eggerthella species (B=14.83, FDR-adjusted p-value=0.04) distinct from Eggerthella lenta, and Lachnospiraceae bacterium 2 1 46FAA (B=115.80, FDR-adjusted p-value=2.84×10⁻¹⁰) whose role is less well-understood, and the association with host inflammation has not been described before. These results highlight the various bacterial species and associated pathogenic functions by which altered gut microbiomes can contribute to inflammaging.

Aging is a common risk factor that often predisposes elderly to the development of neurological disorders such as Parkinson's and Alzheimer's disease. Many studies have shown that Vitamin B12 is involved in several processes of the nervous system, including the synthesis of myelin and post-injury nerve regeneration, the deficiency of which causes peripheral neuropathy, gait ataxia and physical frailty that are often observed in the elderly. A strong association for serum levels of Vitamin B12 and the bacterial species Streptococcus parasanguinis was found (β=312.51, FDR-adjusted p-value=6.04×10⁻⁹, FIG. 10), strains of which are known to produce Vitamin B12, highlighting the metabolic potential for gut bacteria to support healthy aging. Intriguingly, it was also observed a significant association for blood levels of Vitamin B12 and the abundance of Bacteroides coprocola (B=42.89, FDR-adjusted p-value=1.29×10⁻⁴), even though Bacteroidetes are not common B12 producers and instead compete with the host to capture B12 in the gut, indicating that dietary intake might be an explanation for the observed positive association.

Finally, it was also assessed if markers of frailty and musculoskeletal fitness during ageing can be associated with gut microbial biomarkers after accounting for confounding demographic and clinical factors. Strikingly, it was noted that the abundance of Fusobacterium mortiferum, a rarely studied anaerobe, was strongly associated with higher gait speed (β=19.32, FDR-adjusted p-value=2.58×10⁻⁴, FIG. 9). Additionally, a common source of endodontic infections-Dialister invisus—was associated with lower right-hand grip strength (B=−12.81, FDR-adjusted p-value=0.03, FIG. 9), but not left-hand grip strength (as expected from hand dominance, FIG. 9). As Dialister species rely on non-carbohydrate carbon sources for growth such as amino acids and peptides, this observation together with the enrichment of Alistipes species which have multiple putrefaction pathways, highlights the importance of alternate carbon sources in shaping microbial ecology in the aging gut.

### DISCUSSION

The complexity of the human gut microbiome, both in terms of its genetic diversity and the myriad host-microbial interactions that shape it, necessitates that association studies be done with sufficiently high-resolution and power to help infer meaningful insights about function and mechanisms. While prior studies were primarily limited by their resolution (16S rRNA surveys), recent studies have underscored the utility of shotgun metagenomics for species-level and functional pathway analysis, albeit in limited-sized cohorts. By generating deep shotgun metagenomic data for >200 elderly subjects, this study significantly enhances our ability to explore the role of the microbiome in healthy aging. In particular, we provide the first well-powered species-level view of microbiome shifts as a function of age, with a steady decrease in species richness (FIGS. 1B, 1D). This is consistent with a recent report on ‘microbiome uniqueness’ being a marker of healthy aging, but further defines that this is due to an overall shift of microbial community composition with age, with specific species serving as markers (e.g. A. onderdonkii, B. xylanisolvens, R. obeum and E. rectale). Our analysis also highlights that the age-associated shift is on an axis of variation (FIGS. 1B-1D) that is distinct from the axis separating different gut microbiome clusters reminiscent of enterotype structures (see FIG. 12A). In addition, while the overall reduction in microbial diversity observed here (FIG. 1D) is broadly consistent with other studies, this study clarifies that this is primarily due to an increase in uniqueness of microbiome composition and a loss of species richness (see FIGS. 13B, 12B-D), defined in part by several classical butyrate producers in the gut (e.g. F. prausnitzii, R. inulinivorans and E. rectale). It was not observe a general loss in core Bacteroides species as reported in Wilmanski et al, which could be a function of our Asian cohorts, but may also be due to methodological differences (e.g. covariate adjustment). No associations were detected with viruses or fungi, though this could in part be due to their low abundances in the healthy human gut microbiome.

In order to have data from a wide-range of ages and a large number of subjects to sufficiently power our age-association analysis, data from multiple Asian cohorts (SG90, T2D, SPMP, CPE) were combined with metagenomic sequencing data, and attempted to account for differences in how the data was generated using a batch-correction approach (see Table 6). In addition, the association analysis accounted for several demographic and clinical factors that could be confounders, including gender, body mass index, fasting blood glucose, triglyceride, total cholesterol, high density lipoprotein and low-density lipoprotein levels. Nevertheless, it is important to note that batch-correction methods cannot be expected to fully account for all technical sources of variation, and similarly, other potential confounders (e.g. diet, medications) that could not be accounted for as this data was not consistently available across cohorts. Multi-cohort association analysis thus necessarily has to evaluate the robustness of the results obtained as a function of various data subsets used, technical choices made (e.g. normalization) and statistical approaches used. The experiments here suggest that the batch-correction and GLM-based association analysis reported here provide robust results that are reproduced across a majority of conditions tested (see FIG. 11). In addition, this analysis highlights the importance of having sufficient data from older age groups (see FIG. 14). Overall, despite all analytical care, the interpretation of microbiome associations with aging needs care as it is a multi-factorial phenomenon, where for example increased lifetime use of antibiotics could explain loss in gut microbial species richness with age, while at the same time such observations could arise from other factors such as lifestyle choices (e.g. smoking) and their independent impact on longevity and the gut microbiome.

TABLE 6
Experimental methods used in different cohorts for DNA extraction and sequencing

| | Sample | | Library | | Median |
|---|---|---|---|---|---|
| Cohort | collection | DNA Extraction kit | Preparation kit | Sequencer | sequencing depth |
| SG90 | Frozen stool | PowerSoil DNA | Gene Read DNA | Illumina HiSeq X | 8.5M |
| | samples | Isolation Kit | Library I Core Kit | | |
| SPMP | Frozen stool | QIAamp Power Fecal | NEBNext Ultra | Illumina | 36.7M |
| | samples | Pro DNA kit | DNA Library | HiSeq4K | |
| | | | Prep Kit | | |
| CPE | Frozen stool | PowerSoil DNA | Gene Read DNA | Illumina HiSeq | 24M |
| | samples | Isolation Kit | Library I Core Kit | 2500 | |
| T2D | Frozen stool | Custom method¹ | Custom method¹ | Illumina GA | 37.7M |
| | samples | | | | |

¹Qin, J. et al. A metagenome-wide association study of gut microbiota in type 2 diabetes. Nature 490, 55-60 (2012).

The importance of incorporating age and other covariates in microbiome association analyses is important. While this also necessitates larger cohort sizes and extensive metadata collection, a potential advantage is that the few associations that survive covariate adjustments are likely to be more meaningful (e.g. no associations were detected for sleep duration or free thyroxine levels. In combination with shotgun metagenomics, where specific species and functional genes/pathways can be directly quantified, this provides for a powerful avenue for developing meaningful mechanistic hypotheses informed by in vivo human data. In particular, a striking feature of these results is the depletion of a ‘microbial guild’ of butyrate producing species in elderly subjects and its replacement with specific Alistipes species that can produce butyrate through an alternate pathway using amino acids as precursors. This is in agreement with prior studies that have shown reduction in short-chain fatty acid (SCFA) levels, particularly butyrate, in stool samples from the elderly, and association between butyrate levels and frailty, and a hypothesized shift towards bacterial protein fermentation due to increased intestinal transit time. Consistent with this hypothesis, it was also observed a relative reduction of microbial to host reads in elderly subjects (see FIG. 21) that could reflect a reduced capability to support gut microbial fermentation, though direct measurements are needed to confirm this indirect evidence. Changes in dietary patterns (e.g. low fiber) as well as intestinal physiology (e.g. muscle loss, hypochlorhydria) could thus partly explain the observed shift in species composition and deserves further investigation.

Based on the observations here, it is hypothesized that alternate pathways for butyrate production, including using glutarate, 4-amino butyrate and lysine as precursors, may partially compensate for the pyruvate-dependent pathway in gut microbiomes of elderly individuals. Lysine is an essential amino acid that is found in lower plasma concentrations in frail elderly subjects, and lysine supplementation is being investigated in multiple clinical trials for its health benefits. Its use as a supplement to promote healthy aging could thus be another avenue to explore via the pathway of boosting butyrate production in the gut. Among other pathways that were relatively depleted in the elderly, reduction in pyruvate fermentation to acetate and lactate and lipid metabolism pathways (FIG. 3) may have detrimental effects for host health and can be targets for compensation through supplements and probiotics. On the other hand, the relative enrichment of vitamin synthesis pathways in the elderly, including thiamine diphosphate (vitamin B₁), flavin (vitamin B₂), menaquinol (vitamin K₂) and phospho-pantothenate (vitamin B₅), as well as the association of specific species with serum vitamin B₁₂ levels (FIG. 10), could contribute to healthy aging.

Several aging-related chronic diseases, including type 2 diabetes, cardiovascular diseases, and neurological conditions have been linked with the gut microbiome and thus it is essential to account for age as a confounding factor when studying gut microbial mechanisms that contribute to disease. It is hypothesized that having individuals from as wide a range of ages as possible, particularly those who have aged well, will likely help strengthen and refine such association analysis. This is demonstrated in our results for microbiome associations with blood glucose levels where correcting for age avoided potentially spurious associations (e.g. with E. coli). Despite the extensive study of Bifidobacterium species for glycemic control, any significant associations at the genus or species level was not detected suggesting that any in vivo effects for Bifidobacterium might be modulated by other host factors. In contrast, it is noted a significant association for P. goldsteinii, a potential new probiotic species with emerging evidence for diet-dependent role in obesity and type 2 diabetes, though the mechanisms for this effect in mouse models remain to be elucidated.

Systemic low-levels of inflammation are believed to contribute to various aging-related phenotypes (inflammaging). Correspondingly, associations between gut microbial species and markers of inflammation (e.g. hs-CRP and AST) are of particular interest. While the association of a common-source of liver abscesses in Asia (K. pneumoniae) with a liver disease marker (AST), and common oral bacteria (S. infantarius and S. salivarius) with a systemic inflammation marker (hs-CRP) are consistent with disease biology, the distinctness of these associations is striking i.e., while K. pneumoniae is the only species associated with AST, a more diverse group of bacteria are associated with hs-CRP levels. Furthermore, relatively little is known about the two Lachnospiraceae species with strong associations to cholesterol/LDL and hs-CRP levels, respectively, though whole-genome comparisons revealed that the two species are highly diverged (average nucleotide identity <70%) and thus may not have concordant biological roles in the gut microbiome. Similarly, while E. cloacae is an opportunistic pathogen associated with urinary tract and respiratory tract infections in immunocompromised individuals, association between LDL levels and its gut colonization is an interesting link that has not been described before. Further afield, the species level associations reported here for physical frailty markers such as gait speed (F. mortiferum) and grip strength (D. invisus) appear to be distinct from those reported earlier⁹⁶, primarily revolving around the role of butyrate producers in the gut-muscle axis. Together with the strong association of microbial biomarkers such as S. parasanguinis and B. coprocola with serum Vitamin B₁₂ levels, these findings could help develop a non-invasive frailty test based on at-home sample collection.

Overall, these results highlight the value of species-level shotgun metagenomic analysis in large well-characterized cohorts, where further studies in other populations would help determine if some of these associations are specific to Asian lifestyles.

Ageing is associated with chronic diseases resulting in reduced or impaired organ functions. Age is thus a potential confounder since it poses a significant risk for chronic diseases. To avoid such age-related confounding, age was disentangled to identify microbial associations with inflammation, musculoskeletal fitness, and metabolic disease risks. For glucose control, Parabacteroides goldsteinii were identified to associate significantly with increased fasting blood glucose level that were independent from the confounding effects of age, see FIG. 5.

Lachnospiraceae bacterium 14 56FAA and Ruminococcus lactaris were identified to associate with lipid cholesterol controls, see FIG. 6A. Analysis with LDL levels revealed that Lachnospiraceae species, Ruminococcus lactaris and Enterobacter cloacae were negatively associated with LDL levels, see FIG. 6B.

To model microbiome to predict inflammation and sign of infection, taxa association with high-sensitivity C-reactive protein (HSCRP) was analysed. HSCRP is a common marker for inflammation—high level of HSCRP suggests a strong sign of infection. A concerted increased in abundance of Lachnospiraceae bacterium 2 1 46FAA, Streptococcus infantarius, Streptococcus salivarius, Eggerthella unclassified and Escherichia coli, which associated significantly with high level of HSCRP, was observed-see FIG. 7.

Another opportunistic pathogen Klebsiella pneumonia was also associated with the marker for hepatic disease. The ratio of serum Aspartate Aminotransferase (AST) and Alanine Aminotransferase (ALT) are commonly measured clinical biomarkers for monitoring liver injury. A significantly higher abundance of Klebsiella pneumoniae with increased level of AST was observed (FIG. 8A). Klebsiella pneumoniae was also associated with lower ALT level (FIG. 8B).

Gait speed and hand grip strengths were also assessed as frailty markers for mobility and musculoskeletal fitness during ageing. Fast gait speed is determined across a distance of 6 m, which is marked on the floor with tape. Subjects are allowed to use their usual walking aid. Two trials are administered, time (in seconds) is recorded for each trial. Hand grip strength was measured using dynamometry. For mobility, it was found that the abundance of Fusobacterium mortiferum to associate significantly with higher gait speed (FIG. 9A). For hand strengths, Dialister invisus was associated significantly with reduced right-hand grip strength, see FIG. 9B.

Aging is a common risk factor that often predisposes elderly to the development of neurological disorders such as Parkinson's and Alzheimer's disease. Many studies have shown that Vitamin B12 is involved in several processes of the nervous system, including the synthesis of myelin and post-injury nerve regeneration, the deficiency of which causes peripheral neuropathy, gait ataxia and physical frailty that are often observed in the elderly. The strong association of microbial biomarkers such as S. parasanguinis and B. coprocola with serum Vitamin B12 levels could help develop a non-invasive frailty test based on at-home sample collection. The associations of species with Vitamin B12 were assessed and it was found that Streptococcus parasanguinis and Bacteroides coprocola to be positively associated with the levels of Vitamin B12 (see FIG. 10).

Whilst there has been described in the foregoing description preferred embodiments of the present invention, it will be understood by those skilled in the technology concerned that many variations or modifications in details of design or construction may be made without departing from the present invention.

1. A method of determining healthy aging in an individual, the method comprising:
(a) determining the presence or an amount of at least one microorganism in a sample representing the individual's gut microbiome, wherein the at least one microorganism selected from Table 1A, wherein an enrichment of said microorganisms is indicative of healthy aging; and/or
(b) determining the presence or an amount of at least one microorganism in a sample representing the individual's gut microbiome, wherein the at least one microorganism selected from Table 1B, wherein a depletion of said microorganisms is indicative of healthy aging.

2. A method of determining total cholesterol levels in an individual, the method comprising determining the presence or an amount of microorganisms in a sample representing the individual's gut microbiome, wherein the microorganisms are Lachnospiraceae bacterium 14 56FAA and Ruminococcus lactaris, wherein an enrichment of said microorganisms is indicative of a higher than normal level of total cholesterol levels.

3. A method of determining inflammation associated with high-sensitivity C-reactive protein (HSCRP) in an individual, the method comprising determining the presence or an amount of microorganisms in a sample representing the individual's gut microbiome, wherein the microorganisms are Lachnospiraceae bacterium 2 1 46FAA, Streptococcus infantarius, Streptococcus salivarius, Eggerthella unclassified and Escherichia coli, wherein an enrichment of said microorganisms is indicative of inflammation.

4. A method of determining hepatic health in an individual, the method comprising determining the presence or an amount of Klebsiella pneumoniae in a sample representing the individual's gut microbiome, wherein an enrichment of said microorganism is indicative of hepatic disease.

5. (canceled)

6. (canceled)

7. A method of determining if a subject has healthy levels of vitamin B12, the method comprising determining the presence or an amount of Streptococcus parasanguinis or Bacteroides coprocola in a sample representing the individual's gut microbiome, wherein an enrichment of said microorganism is indicative of healthy levels of vitamin B12.

8. A method of promoting healthy ageing in a subject, the method comprising administering to a patient a composition according to claim 10 that improves intestinal flora by:
(a) enriching at least one microorganism selected from Table 1A, and/or
(b) reducing or suppressing at least one microorganism selected from Table 1B.

9. (canceled)

10. A composition or pharmaceutical composition for promoting healthy ageing, the composition comprising an agent for:
(a). enriching at least one microorganism selected from Table 1A, and/or
(b). reducing or suppressing at least one microorganism selected from Table 1B.

11. (canceled)

12. A method of determining the likelihood of healthy aging in an individual, the method comprising:
(a) determining a gut microbiome signature of the individual based on the presence or amount of the at least one microorganism determined in step (a) and/or step (b) of claim 1; and
(b) applying a prediction model to assess the gut microbiome signature with respect to a gut profile representative of an individual that is aging healthily, wherein the prediction model is trained using a dataset of microbiome profiles of a plurality of individuals who are aging healthily and said gut profile comprises:
(i) an enrichment of at least one microorganism selected from Table 1A; and/or
(ii) a depletion of at least one microorganism selected from Table 1B.

13. The method according to claim 12, wherein the prediction model comprises a machine learning probability model.

14. A computer readable storage medium comprising computer readable instructions operable when executed by a computer to predict the likelihood of healthy aging in an individual, the computer readable instructions configured to perform a method according to claim 12.

15. An apparatus or system comprising:
(a) a receiving unit configured to receive a dataset of values representing a gut microbiome signature of an individual by determining an amount of, or presence or absence of, each microorganism in a group of microorganisms present in a sample obtained from the individual; and
(b) a processor configured to process a prediction model to assess the gut microbiome signature with respect to a gut profile representative of good gut health to obtain a likelihood of healthy aging in the individual,
wherein the prediction model is trained using a dataset of microbiome profiles of a plurality of individuals who are aging healthily and said profile comprises:
(i) an enrichment of at least one microorganism selected from Table 1A; and/or
(ii) a depletion of at least one microorganism selected from Table 1B.

16. An assay kit for use in the method according to claim 1, additionally comprising instructions to be used in the method.
